(19) United States
(12) Patent Application Publication
(10) Pub. No.: US 2010/0173914 A1
(43) Pub. Date: Jul. 8, 2010

(54) PHARMACEUTICALS, COMPOSITIONS AND METHODS OF MAKING AND USING THE SAME

(76) Inventors: Hexi Chang, Belmont, MA (US); Benjamin Lane, Chelsea, MA (US); Weirong Chen, Waltham, MA (US); Slawomir Janicki, North Chelmsford, MA (US); Richard Todd, Winnersh (GB); William F. Kiesman, Wayland, MA (US)

Correspondence Address: STEPTOE & JOHNSON LLP, 1330 CONNECTICUT AVE., NW, WASHINGTON, DC, 20036, US

(21) Appl. No.: 12/522,995

(22) PCT Filed: Jan. 2, 2008
(86) PCT No.: PCT/US08/50027

§ 371 Date: Feb. 18, 2010

Related U.S. Application Data
(60) Provisional application No. 60/884,746, filed on Jan. 12, 2007.

(51) Int. Cl. A61K 31/519; C07D 487/04; A61P 25/16; A61P 25/14; A61P 25/00; A61P 25/24; A61P 25/28

(52) U.S. Cl. 514/261.1; 544/254

(57) ABSTRACT

Compounds that are capable of acting as purine receptor antagonists, pharmaceutical compositions including the compounds, and methods of making the compounds, are disclosed. The compounds and compositions can be used in treating or preventing disorders related to purine receptor hyperfunctioning.

### CLAIM OF PRIORITY

This application claims priority to provisional U.S. Patent Application No. 60/884,746, filed Jan. 12, 2007, and titled “Pharmaceuticals, Compositions, and Methods of Making and Using the Same,” and which is incorporated by reference in its entirety.

### TECHNICAL FIELD

The present invention relates to pharmaceutical compositions and methods, and methods of making and using the same.

### BACKGROUND

Movement disorders constitute a serious health problem, especially among the elderly. These movement disorders can often be the result of brain lesions. Disorders involving the basal ganglia which result in movement disorders include Parkinson's disease, Huntington's chorea and Wilson's disease. Furthermore, dyskinesias often arise as sequelae of cerebral ischaemia and other neurological disorders.

There are four classic symptoms of Parkinson's disease: tremor, rigidity, akinesia and postural changes. The disease is also commonly associated with depression, dementia and overall cognitive decline. Parkinson's disease has a prevalence of 1 per 1,000 of the total population. The incidence increases to 1 per 100 for those aged over 60 years. Degeneration of dopaminergic neurons in the substantia nigra and the subsequent reductions in interstitial concentrations of dopamine in the striatum are critical to the development of Parkinson's disease. Some 80% of cells from the substantia nigra can be destroyed before the clinical symptoms of Parkinson's disease become apparent.

Some strategies for the treatment of Parkinson's disease are based on transmitter replacement therapy (L-dihydroxyphenylacetic acid (L-DOPA)), inhibition of monoamine oxidase (e.g., Deprenyl™), dopamine receptor agonists (e.g., bromocriptine and apomorphine) and anticholinergics (e.g., benztrophine, orphenadrine). Transmitter replacement therapy may not provide consistent clinical benefit, especially after prolonged treatment when “on-off” symptoms develop. Furthermore, such treatments have also been associated with involuntary movements of athetosis and chorea, nausea and vomiting. Additionally, current therapies do not treat the underlying neurodegenerative disorder resulting in a continuing cognitive decline in patients.

### SUMMARY

Blocking of purine receptors, particularly adenosine receptors, and more particularly adenosine A_{2A} receptors may be beneficial in treatment or prevention of movement disorders such as Parkinson's disease, or disorders such as depression, cognitive, or memory impairment, acute and chronic pain, ADHD or narcolepsy, or for neuroprotection in a subject.

In one aspect, a compound or a pharmaceutically acceptable salt thereof has formula (I):

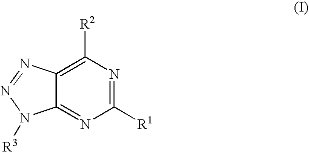

R¹ can be selected from H, alkyl, aryl, heteroaryl, cycloalkyl, heterocyclyl, alkoxy, aryloxy, heteroaryloxy, alkylthio, arylthio, heteroarylthio, halogen, —CN, —NR⁵R⁶, —N(R^{a})C(O)R⁴, —N(R^{a})C(O)NR⁵R⁶, —N(R^{a})CO₂R⁴, and —N(R^{a})SO₂R⁴.

R² can be aryl optionally substituted by 1-3 substituents selected from R⁷, or heteroaryl optionally substituted by 1-3 substituents selected from R⁷.

R³ can have the formula -L-Ar³-N(R^{a})SO₃—R^{b}, where L is a bond, -(CR^{a}R^{b})ₙ—, —C(O)—, —C(O)N(R^{a})—, -(CR^{a}R^{b})ₙ—C(O)N(R^{a})—, —C(O)N(R^{a})—(CR^{a}R^{b})ₙ—, —(CR^{a}R^{b})ₙ—O—. Ar³ can be arylene optionally substituted by 1-3 substituents selected from R⁷, or heteroarylene optionally substituted by 1-3 substituents selected from R⁷.

Each R⁴ can be, independently, H, alkyl, or aryl, where alkyl and aryl are each independently substituted by 1-3 substituents selected from R⁷.

Each R⁵ and each R⁶ can be, independently, H, alkyl or aryl where alkyl and aryl are each independently substituted by 1-3 substituents selected from R⁷. Alternatively, R⁵ and R⁶ together with the atom to which they are attached can form a heterocyclic group which is optionally substituted by 1-3 substituents selected from R⁷.

Each R⁷, independently, can be H, oxo, CN, halogen, —CF₃, —CHF₂, —CHO, —OH, —NO₂, —SH, —OCF₃, alkyl, alkoxy, alkenyl, alkynyl, cycloalkyl, aryl, heteroaryl, heterocyclyl, —CO₂R^{a}, —O-alkyl, —O-alkenyl, —O-alkynyl, —O-cycloalkyl, —O-aryl, —O-heteroaryl, —O-heterocyclyl, —(CH₂)-alkyl, —(CH₂)-alkoxy, —(CH₂)ₙ-alkenyl, —(CH₂)ₙ-alkynyl, —(CH₂)-cycloalkyl, —(CH₂)-aryl, —(CH₂)-heteroaryl, —(CH₂)ₙ-heterocyclyl, —N(R^{a})-alkyl, —N(R^{a})-alkoxy, —N(R^{a})-alkenyl, —N(R^{a})-alkynyl, —N(R^{a})-cycloalkyl, —N(R^{a})-aryl, —N(R^{a})-heteroaryl, —N(R^{a})-heterocyclyl, —SOₘ-alkyl, —SOₘ-alkoxy, —SOₘ-alkenyl, —SOₘ-alkynyl, —SOₘ-cycloalkyl, —SOₘ-aryl, —SOₘ-heteroaryl, —SOₘ-heterocyclyl, —N(R^{a})C(O)-alkyl, —N(R^{a})C(O)-alkoxy, —N(R^{a})C(O)-alkenyl, —N(R^{a})C(O)-alkynyl, —N(R^{a})C(O)-cycloalkyl, —N(R^{a})C(O)-aryl, —N(R^{a})C(O)-heteroaryl, —N(R^{a})C(O)-heterocyclyl, —C(O)N(R^{a})-alkyl, —C(O)N(R^{a})-alkoxy, —C(O)N(R^{a})-alkenyl, —C(O)N(R^{a})-alkynyl, —C(O)N(R^{a})-cycloalkyl, —C(O)N(R^{a})-aryl, —C(O)N(R^{a})-heteroaryl, or —C(O)N(R^{a})-heterocyclyl;

Each R^{a}, independently, can be H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl.

Each R^{b}, independently, can be H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl.

Each m, independently, can be 0, 1, or 2; and each n, independently, can be 0, 1, 2, 3, or 4.

In some circumstances, R¹ can be —NR⁵R⁶, or —NH₂. R² can be furyl, thienyl, imidazolyl, phenyl, pyridyl, thiazolyl, pyrazolyl, triazolyl, pyrrolyl, or oxazolyl, each of which is optionally substituted by 1-3 substituents selected from R⁷. R² can be furyl, thienyl, phenyl, methylfuryl, or methoxyphenyl. In some circumstances, L can be —CH₂— and Ar³ can be arylene, such as phenylene, methylphenylene, or methoxyphenylene.

In some circumstances, R¹ can be —NR⁵R⁶, R² can be furyl, thienyl, imidazolyl, phenyl, pyridyl, thiazolyl, pyrazolyl, triazolyl, pyrrolyl, or oxazolyl (each of which is optionally substituted by 1-3 substituents selected from R⁷), L can be —CH₂—, and Ar³ can be arylene optionally substituted by 1-3 substituents selected from R⁷. For example, R¹ can be —NH₂, R² can be furyl, thienyl, phenyl, methylfuryl, or methoxyphenyl, L can be —CH₂—, and Ar³ can be phenylene, methylphenylene, or methoxyphenylene.

The compound can be selected from the group consisting of 4-[(5-amino-7-(furan-2-yl)-3H-[1,2,3]triazolo[4,5-d]pyrimidin-3-yl)methyl]-2-methylphenylsulfamic acid; 4-[(5-amino-7-(furan-2-yl)-3H-[1,2,3]triazolo[4,5-d]pyrimidin-3-yl)methyl]phenylsulfamic acid; 4-[(5-amino-7-phenyl,2,3]triazolo[4,5-d]pyrimidin-3-yl)methyl]phenylsulfamic acid; 4-[(5-amino-7-(thiophen-2-yl)-3H-[1,2,3]triazolo[4,5-d]pyrimidin-3-yl)methyl]phenylsulfamic acid; 4-[(5-amino-7-(2-methoxyphenyl)-3H-[1,2,3]triazolo[4,5-d]pyrimidin-3-yl)methyl]phenylsulfamic acid; 3-[(5-amino-7-(furan-2-yl)-3H-[1,2,3]triazolo[4,5-d]pyrimidin-3-yl)methyl]-phenylsulfamic acid; 3-[(5-amino-7-(thiophen-2-yl)-3H-[1,2,3]triazolo[4,5-d]pyrimidin-3-yl)methyl]-phenylsulfamic acid; 3-[(5-amino-7-(2-methoxyphenyl)-3H-[1,2,3]triazolo[4,5-d]pyrimidin-3-yl)methyl]-phenylsulfamic acid; 3-[(5-amino-7-phenyl-3H-[1,2,3]triazolo[4,5-d]pyrimidin-3-yl)methyl]-phenylsulfamic acid; 4-[(5-amino-7-(2-methoxyphenyl)-3H-[1,2,3]triazolo[4,5-d]pyrimidin-3-yl)methyl]-2-methylphenylsulfamic acid; 4-[(5-amino-7-(thiophen-2-yl)-3H-[1,2,3]triazolo[4,5-d]pyrimidin-3-yl)methyl]-2-methylphenylsulfamic acid; 4-[(5-amino-7-phenyl-3H-[1,2,3]triazolo[4,5-d]pyrimidin-3-yl)methyl]-2-methylphenylsulfamic acid; 5-[(5-amino-7-(thiophen-2-yl)-3H-[1,2,3]triazolo[4,5-d]pyrimidin-3-yl)methyl]-2-methylphenylsulfamic acid; 5-[(5-amino-7-(furan-2-yl)-3H-[1,2,3]triazolo[4,5-d]pyrimidin-3-yl)methyl]-2-methylphenylsulfamic acid; 5-[(5-amino-7-phenyl-3H-[1,2,3]triazolo[4,5-d]pyrimidin-3-yl)methyl]-2-methylphenylsulfamic acid; 5-[(5-amino-7-(2-methoxyphenyl)-3H-[1,2,3]triazolo[4,5-d]pyrimidin-3-yl)methyl]-2-methylphenylsulfamic acid; 4-[(5-amino-7-(2-methoxyphenyl)-3H-[1,2,3]triazolo[4,5-d]pyrimidin-3-yl)methyl]-2-methoxyphenylsulfamic acid; 4-[(5-amino-7-phenyl-3H-[1,2,3]triazolo[4,5-d]pyrimidin-3-yl)methyl]-2-methoxyphenylsulfamic acid; 4-[(5-amino-7-(thiophen-2-yl)-3H-[1,2,3]triazolo[4,5-d]pyrimidin-3-yl)methyl]-2-methoxyphenylsulfamic acid; 4-[(5-amino-7-(furan-2-yl)-3H-[1,2,3]triazolo[4,5-d]pyrimidin-3-yl)methyl]-2-methoxyphenylsulfamic acid; 4-[(5-amino-7-(thiophen-2-yl)-3H-[1,2,3]triazolo[4,5-d]pyrimidin-3-yl)methyl]-3-methoxyphenylsulfamic acid; 4-[(5-amino-7-(furan-2-yl)-3H-[1,2,3]triazolo[4,5-d]pyrimidin-3-yl)methyl]-3-methoxyphenylsulfamic acid; 4-[(5-amino-7-phenyl-3H-[1,2,3]triazolo[4,5-d]pyrimidin-3-yl)methyl]-3-methoxyphenylsulfamic acid; 4-[(5-amino-7-(2-methoxyphenyl)-3H-[1,2,3]triazolo[4,5-d]pyrimidin-3-yl)methyl]-3-methoxyphenylsulfamic acid; 4-[(5-amino-7-(5-methylfuran-2-yl)-3H-[1,2,3]triazolo[4,5-d]pyrimidin-3-yl)methyl]phenylsulfamic acid; 3-[(5-amino-7-(5-methylfuran-2-yl)-3H-[1,2,3]triazolo[4,5-d]pyrimidin-3-yl)methyl]phenylsulfamic acid; 5-[(5-amino-7-(5-methylfuran-2-yl)-3H-[1,2,3]triazolo[4,5-d]pyrimidin-3-yl)methyl]-2-methylphenylsulfamic acid; 4-[(5-amino-7-(5-methylfuran-2-yl)-3H-[1,2,3]triazolo[4,5-d]pyrimidin-3-yl)methyl]-2-methylphenylsulfamic acid; 4-[(5-amino-7-(5-methylfuran-2-yl)-3H-[1,2,3]triazolo[4,5-d]pyrimidin-3-yl)methyl]-2-methoxyphenylsulfamic acid; 4-[(5-amino-7-(5-methylfuran-2-yl)-3H-[1,2,3]triazolo[4,5-d]pyrimidin-3-yl)methyl]-3-methoxyphenylsulfamic acid; and pharmaceutically acceptable salts thereof.

In another aspect, a pharmaceutical composition includes a pharmaceutically acceptable carrier and a compound of formula (I).

In another aspect, a method of treating a disorder includes administering an effective dose of a compound of formula (I), or a pharmaceutically acceptable salt thereof, to a subject in need of treatment of a disorder treatable by purine receptor blocking.

The disorder can be related to hyper functioning of purine receptors. The subject can be in need of adenosine receptor blocking. The adenosine receptors can be A_{2A} receptors. The disorder can be a movement disorder. The movement disorder can be Parkinson's disease; or the movement disorder can be drug-induced Parkinsonism, post-encephalitic Parkinsonism, Parkinsonism induced by poisoning or post-traumatic Parkinson's disease. The movement disorder can be progressive supernuclear palsy, Huntington's disease, multiple system atrophy, corticobasal degeneration, Wilson's disease, Hallerrorden-Spatz disease, progressive pallidal atrophy, Dopa-responsive dystonia-Parkinsonism, spasticity or other disorders of the basal ganglia which result in dyskinesias.

The method can include administering to the subject an additional drug useful in the treatment of movement disorders. The additional drug useful in the treatment of movement disorders can be a drug useful in the treatment of Parkinson's disease, such as, for example, L-DOPA or a dopamine agonist. The disorder can be depression, a cognitive or memory impairment disorder, acute or chronic pain, ADHD or narcolepsy. The cognitive or memory impairment disorder can be Alzheimer's disease.

In another aspect, a method of making of compound includes contacting a dithionite salt with a compound having the formula (I):

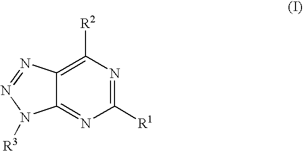

where R¹ and R² are as defined above, and R³ has the formula -L-Ar³-NO₂, wherein L is a bond, —(CR^{a}R^{b})ₙ—, —C(O)—, —C(O)N(R^{a})—, —(CR^{a}R^{b})ₙ—C(O)N(R^{a})—, —C(O)N(R^{a})—(CR^{a}R^{b})ₙ—, —(CR^{a}R^{b})ₙ—O—; and wherein Ar³ is arylene optionally substituted by 1-3 substituents selected from R⁷, or heteroarylene optionally substituted by 1-3 substituents selected from R⁷, and R⁷ is as defined above. The dithionite salt can be sodium dithionite (Na₂S₂O₄).

In another aspect, a method of making of compound includes contacting chlorosulfonic acid with a compound having the formula (I):

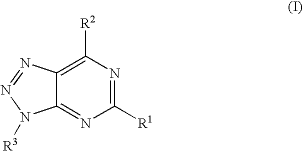

where R¹ and R² are as defined above, and R³ has the formula -L-Ar³—NH₂, wherein L is a bond, —(CR^{a}R^{b})ₙ—, —C(O)—, —C(O)N(R^{a})—, —(CR^{a}R^{b})ₙ—C(O)N(R^{a})—, —C(O)N(R^{a})—(CR^{a}R^{b})ₙ—, —(CR^{a}R^{b})ₙ—O—; and wherein Ar³ is arylene optionally substituted by 1-3 substituents selected from R⁷, or heteroarylene optionally substituted by 1-3 substituents selected from R⁷, and R⁷ is as defined above.

Other aspects, features, and objects will be apparent from the description and drawings.

### DETAILED DESCRIPTION

Blockade of A₂ adenosine receptors has been implicated in the treatment of movement disorders such as Parkinson's disease and in the treatment of cerebral ischemia. See, for example, WO 02/055083; Richardson, P. J. et al., Trends Pharmacol. Sci. 1997, 18, 338-344; and Gao, Y. and Phillis, J. W., Life Sci. 1994, 55, 61-65, each of which is incorporated by reference in its entirety. Adenosine A_{2A} receptor antagonists have potential use in the treatment of movement disorders such as Parkinson's Disease (Mally, J. and Stone, T. W., CNS Drugs, 1998, 10, 311-320, which is incorporated by reference in its entirety).

Adenosine is a naturally occurring purine nucleoside which has a wide variety of well-documented regulatory functions and physiological effects. The central nervous system (CNS) effects of this endogenous nucleoside have attracted particular attention in drug discovery, because of the therapeutic potential of purinergic agents in CNS disorders (Jacobson, K. A. et al., J. Med. Chem. 1992, 35, 407-422, and Bhagwhat, S. S.; Williams, M. E. Opin. Ther. Patents 1995, 5,547-558, each which is incorporated by reference in its entirety).

Adenosine receptors represent a subclass (P₁) of the group of purine nucleotide and nucleoside receptors known as purinoreceptors. The main pharmacologically distinct adenosine receptor subtypes are known as A₁, A_{2A}, A_{2B} (of high and low affinity) and A₃ (Fredholm, B. B., et al., Pharmacol. Rev. 1994, 46, 143-156, which is incorporated by reference in its entirety). The adenosine receptors are present in the CNS (Fredholm, B. B., News Physiol. Sci., 1995, 10, 122-128, which is incorporated by reference in its entirety).

P₁ receptor-mediated agents can be useful in the treatment of cerebral ischemia or neurodegenerative disorders, such as Parkinson's disease (Jacobson, K. A., Suzuki, F., Drug Dev. Res., 1997, 39, 289-300; Baraldi, P. G. et al., Curr. Med. Chem. 1995, 2, 707-722; and Williams, M. and Bumnstock, G. Purinergic Approaches Exp. Ther. (1997), 3-26. Editor. Jacobson, Kenneth A.; Jarvis, Michael F. Publisher: Wiley-liss, New York, N.Y., which is incorporated by reference in its entirety).

It has been speculated that xanthine derivatives such as caffeine may offer a form of treatment for attention-deficit hyperactivity disorder (ADHD). A number of studies have demonstrated a beneficial effect of caffeine on controlling the symptoms of ADHD (Garfinkel, B. D. et al., Psychiatry, 1981, 26, 395-401, which is incorporated by reference in its entirety). Antagonism of adenosine receptors is thought to account for the majority of the behavioral effects of caffeine in humans and thus blockade of adenosine A_{2A} receptors may account for the observed effects of caffeine in ADHD patients. Therefore a selective adenosine A_{2A} receptor antagonist may provide an effective treatment for ADHD but with decreased side-effects.

Adenosine receptors can play an important role in regulation of sleep patterns, and indeed adenosine antagonists such as caffeine exert potent stimulant effects and can be used to prolong wakefulness (Porkka-Heiskanen, T. et al., Science, 1997, 276, 1265-1268, which is incorporated by reference in its entirety). Adenosine's sleep regulation can be mediated by the adenosine A_{2A} receptor (Satoh, S., et al., Proc. Natl. Acad. Sci., USA, 1996, 93: 5980-5984, which is incorporated by reference in its entirety). Thus, a selective adenosine A_{2A} receptor antagonist may be of benefit in counteracting excessive sleepiness in sleep disorders such as hypersomnia or narcolepsy.

Patients with major depression demonstrate a blunted response to adenosine agonist-induced stimulation in platelets, suggesting that a dysregulation of adenosine A_{2A} receptor function may occur during depression (Berk, M. et al., 2001, Eur. Neuropsycopharmacol. 11, 183-186, which is incorporated by reference in its entirety). Experimental evidence in animal models has shown that blockade of adenosine A_{2A} receptor function confers antidepressant activity (El Yacoubi, M et al., Br. J. Pharmacol. 2001, 134, 68-77, which is incorporated by reference in its entirety). Thus, adenosine A_{2A} receptor antagonists may be useful in treatment of major depression and other affective disorders in patients.

The pharmacology of adenosine A_{2A} receptors has been reviewed (Ongini, E.; Fredholm, B. B. Trends Pharmacol. Sci. 1996, 17(10), 364-372, which is incorporated by reference in its entirety). One possible mechanism in the treatment of movement disorders by adenosine A_{2A} antagonists is that A_{2A} receptors may be functionally linked dopamine D₂ receptors in the CNS. See, for example, Ferre, S. et al., Proc. Natl. Acad. Sci. USA 1991, 88, 7238-7241; Puxe, K. et al., Adenosine Adenine Nucleotides Mol. Biol. Integr. Physiol., (Proc. Int. Symp.), 5th (1995), 499-507. Editors: Belardinelr, Luiz; Pelleg, Amir Publisher: Kluwer, Boston, Mass.; and Ferre, S. et al., Trends Neurosci. 1997, 20, 482-487, each of which is incorporated by reference in its entirety.

Interest in the role of adenosine A_{2A} receptors in the CNS, due in part to in vivo studies linking A_{2A} receptors with catalepsy (Ferre et al., Neurosci. Lett. 1991, 130, 1624; and Mandhane, S, N. et al., Eur. J. Pharmacol. 1997, 328, 135-141, each of which is incorporated by reference in its entirety), has prompted investigations into agents that selectively bind to adenosine A_{2A} receptors.

One advantage of adenosine A_{2A} antagonist therapy is that the underlying neurodegenerative disorder may also be treated. See, e.g., Ongini, E.; Adami, M.; Ferri, C.; Bertorelli, R., Ann. N.Y. Acad. Sci. 1997, 825 (Neuroprotective Agents), 3048, which is incorporated by reference in its entirety. In particular, blockade of adenosine A_{2A} receptor function confers neuroprotection against MPTP-induced neurotoxicity in mice (Chen, J-F., J. Neurosci. 2001, 21, RC143, which is incorporated by reference in its entirety). In addition, consumption of dietary caffeine (a known adenosine A_{2A} receptor antagonist), is associated with a reduced risk of Parkinson's disease (Ascherio, A. et al, Ann. Neurol., 2001, 50, 56-63; and Ross G. W., et al., JAMA, 2000, 283, 2674-9, each of which is incorporated by reference in its entirety). Thus, adenosine A_{2A} receptor antagonists may confer neuroprotection in neurodegenerative diseases such as Parkinson's disease.

Xanthine derivatives have been disclosed as adenosine A_{2A} receptor antagonists for treating various diseases caused by hyperfunctioning of adenosine A₂ receptors, such as Parkinson's disease (see, for example, EP-A-565377, which is incorporated by reference in its entirety). One prominent xanthine-derived adenosine A_{2A} selective antagonist is CSC [8-(3-chlorostyryl)caffeine] (Jacobson et al., FEBS Lett., 1993, 323, 141-144, which is incorporated by reference in its entirety).

Theophylline (1,3-dimethylxanthine), a bronchodilator drug which is a mixed antagonist at adenosine A₁ and A_{2A} receptors, has been studied clinically. To determine whether a formulation of this adenosine receptor antagonist would be of value in Parkinson's disease an open trial was conducted on 15 Parkinsonian patients, treated for up to 12 weeks with a slow release oral theophylline preparation (150 mg/day), yielding serum theophylline levels of 4.44 mg/L after one week. The patients exhibited significant improvements in mean objective disability scores and 11 reported moderate or marked subjective improvement (Mally, J., Stone, T. W. J. Pharm. Pharmacol. 1994, 46, 515-517, which is incorporated by reference in its entirety).

KF 17837 (E-8-(3,4-dimethoxystyryl)-1,3-dipropyl-7-methylxanthine) is a selective adenosine A_{2A} receptor antagonist which on oral administration significantly ameliorated the cataleptic responses induced by intracerebroventricular administration of an adenosine A_{2A} receptor agonist, CGS 21680. KF 17837 also reduced the catalepsy induced by haloperidol and reserpine. Moreover, KF 17837 potentiated the anticataleptic effects of a subthreshold dose of L-DOPA plus benserazide, suggesting that KF 17837 is a centrally active adenosine A_{2A} receptor antagonist and that the dopaminergic function of the nigrostriatal pathway is potentiated by adenosine A_{2A} receptor antagonists (Kanda, T. et al., Eur. J. Pharmacol. 1994, 256, 263-268, which is incorporated by reference in its entirety). The structure activity relationship (SAR) of KF 17837 has been published (Shimada, J. et al., Bioorg. Med. Chem. Lett. 1997, 7, 2349-2352, which is incorporated by reference in its entirety). Recent data has also been provided on the adenosine A_{2A} receptor antagonist KW-6002 (Kuwana, Y et al., Soc. Neurosci. Abstr. 1997,23, 119.14; and Kanda, T. et al., Ann. Neurol. 1998,43(4), 507-513, each of which is incorporated by reference in its entirety).

Non-xanthine structures sharing these pharmacological properties include SCH 58261 and its derivatives (Baraldi, P. G. et al., J. Med. Chem. 1996, 39, 1164-71, which is incorporated by reference in its entirety). SCH 58261 (7-(2-phenylethyl)-5-amino-2-(2-furyl)-pyrazolo-[4,3-e]-1,2,4-triazolo[1,5-d]pyrimidine) is reported as effective in the treatment of movement disorders (Ongini, E. Drug Dev. Res. 1997, 42(2), 63-70, which is incorporated by reference in its entirety) and has been followed up by a later series of compounds (Baraldi, P. G. et al., J. Med. Chem. 1998,41(12), 2126-2133, which is incorporated by reference in its entirety).

A number of adenosine A_{2A} antagonists are described in International Patent Application Publication WO 02/055083 A1, which is incorporated by reference in its entirety.

Compounds of formula (I) are useful as purine receptor antagonists, for example, as adenosine A_{2A} antagonists. In particular the compounds can have formula (I) as detailed below:

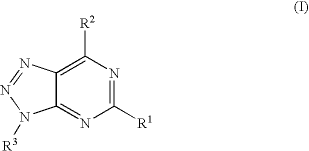

R¹ can be selected from H, alkyl, aryl, heteroaryl, cycloalkyl, heterocyclyl, alkoxy, aryloxy, heteroaryloxy, alkylthio, arylthio, heteroarylthio, halogen, —CN, —NR⁵R⁶, —N(R^{a})C(O)R⁴, —N(R^{a})C(O)NR⁵R⁶, —N(R^{a})CO₂R⁴, and —N(R^{a})SO₂R⁴.

R² can be aryl optionally substituted by 1-3 substituents selected from R⁷, or heteroaryl optionally substituted by 1-3 substituents selected from R⁷.

R³ can have the formula -L-Ar³-N(R^{a})SO₃—R^{b}, where L is a bond, —(CR^{a}R^{b})ₙ—, —C(O)—, —C(O)N(R^{a})—, —(CR^{a}R^{b})ₙ—C(O)N(R^{a})—, —C(O)N(R^{a})—(CR^{a}R^{b})ₙ—, —(CR^{a}R^{b})ₙ—O—. Ar³ can be arylene optionally substituted by 1-3 substituents selected from R⁷, or heteroarylene optionally substituted by 1-3 substituents selected from R⁷.

Each R⁴ can be, independently, H, alkyl, or aryl, where alkyl and aryl are each independently substituted by 1-3 substituents selected from R⁷.

Each R⁵ and each R⁶ can be, independently, H, alkyl or aryl where alkyl and aryl are each independently substituted by 1-3 substituents selected from R⁷. Alternatively, R⁵ and R⁶ together with the atom to which they are attached can form a heterocyclic group which is optionally substituted by 1-3 substituents selected from R⁷.

Each R⁷, independently, can be H, oxo, CN, halogen, —CF₃, —CHF₂, —CHO, —OH, —NO₂, —SH, —OCF₃, alkyl, alkoxy, alkenyl, alkynyl, cycloalkyl, aryl, heteroaryl, heterocyclyl, —CO₂R^{a}, —O-alkyl, —O-alkenyl, —O-alkynyl, —O-cycloalkyl, —O-aryl, —O-heteroaryl, —O-heterocyclyl, —(CH₂)ₙ-alkyl, —(CH₂)ₙ-alkoxy, —(CH₂)ₙ-alkenyl, —(CH₂)ₙ-alkynyl, —(CH₂)ₙ-cycloalkyl, —(CH₂)ₙ-aryl, —(CH₂)ₙ-heteroaryl, —(CH₂)_{b}-heterocyclyl, —N(R^{a})-alkyl, —N(R^{a})-alkoxy, —N(R^{a})-alkenyl, —N(R^{a})-alkynyl, —N(R^{a})-cycloalkyl, —N(R^{a})-aryl, —N(R^{a})-heteroaryl, —N(R^{a})-heterocyclyl, —SOₘ-alkyl, —SOₘ-alkoxy, —SOₘ-alkenyl, —SOₘ-alkynyl, —SOₘ-cycloalkyl, —SOₘ-aryl, —SOₘ-heteroaryl, —SOₘ-heterocyclyl, —N(R^{a})C(O)-alkyl, —N(R^{a})C(O)-alkoxy, —N(R^{a})C(O)-alkenyl, —N(R^{a})C(O)-alkynyl, —N(R^{a})C(O)-cycloalkyl, —N(R^{a})C(O)-aryl, —N(R^{a})C(O)-heteroaryl, —N(R^{a})C(O)-heterocyclyl, —C(O)N(R^{a})-alkyl, —C(O)N(R^{a})-alkoxy, —C(O)N(R^{a})-alkenyl, —C(O)N(R^{a})-alkynyl, —C(O)N(R^{a})-cycloalkyl, —C(O)N(R^{a})-aryl, —C(O)N(R^{a})-heteroaryl, or —C(O)N(R^{a})-heterocyclyl;

Each R^{a}, independently, can be H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl.

Each R^{b}, independently, can be H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl.

Each m, independently, can be 0, 1, or 2; and each n, independently, can be 0, 1, 2, 3, or 4. Pharmaceutically acceptable salts of the compounds of formula (I) as described above are also suitable as purine receptor antagonists, for example, as adenosine A_{2A} antagonists.

As used herein, the term “alkyl,” alone or in combination, refers to a straight-chain or branched-chain alkyl radical containing 1 to 10, 1 to 6, or 1 to 4, carbon atoms. Examples of such radicals include, but are not limited to, methyl, ethyl, n-propyl, isopropyl, n-butyl, isobutyl, sec-butyl, tert-butyl, pentyl, iso-amyl, hexyl, decyl and the like.

The term “alkenyl,” alone or in combination, refers to a straight-chain or branched-chain alkenyl radical containing 2 to 10, 2 to 6, or 2 to 4, carbon atoms. Examples of such radicals include, but are not limited to, ethenyl, E- and Z-propenyl, isopropenyl, E- and Z-butenyl, E- and Z-isobutenyl, E- and Z-pentenyl, decenyl and the like.

The term “alkynyl,” alone or in combination, refers to a straight-chain or branched-chain alkynyl radical containing 2 to 10, 2 to 6, or 2 to 4, carbon atoms. Examples of such radicals include, but are not limited to, ethynyl (acetylenyl), propynyl, propargyl, butynyl, hexynyl, decynyl and the like.

The term “cycloalkyl,” alone or in combination, refers to a cyclic alkyl radical containing 3 to 10, 3 to 8, or 3 to 6, carbon atoms. Examples of such cycloalkyl radicals include, but are not limited to, cyclopropyl, cyclobutyl, cyclopentyl, cyclohexyl, and the like; and bicylic groups including bicyclo[3.4.0]nonyl, bicyclo[2.2.2]octyl, norbornyl, spiro[4.5]decyl, and the like.

The term “cycloalkenyl,” alone or in combination, refers to a cyclic carbocycle containing 4 to 10, 4 to 8, or 5 or 6, carbon atoms and one or more double bonds. Examples of such cycloalkenyl radicals include, but are not limited to, cyclopentenyl, cyclohexenyl, cyclopentadienyl, and bicyclic groups such as norbornenyl, and the like.

The term “aryl” refers to a carbocyclic aromatic group, and includes fused bicyclic or tricyclic systems where one or more rings are not aromatic, e.g., indanyl. Examples of such carbocyclic aromatic groups include, but are not limited to, phenyl, naphthyl, indenyl, indanyl, azulenyl, fluorenyl, and anthracenyl.

The term “heteroaryl” refers to a heterocyclic aromatic group, and includes fused bicyclic or tricyclic systems where one or more rings are not aromatic, e.g., indolinyl. Examples of such heterocyclic aromatic groups include, but are not limited to, furyl, thienyl, pyridyl, pyrrolyl, oxazolyl), thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, carbazolyl, acridinyl, phenazinyl, phenothiazinyl, and phenoxazinyl.

The term “alkoxy,” alone or in combination, refers to an alkyl ether radical, or cycloalkyl ether radical, where the terms “alkyl” and “cycloalkyl” are as defined above. Examples of suitable alkyl ether radicals include, but are not limited to, methoxy, ethoxy, n-propoxy, iso-propoxy, n-butoxy, iso-butoxy, sec-butoxy, tert-butoxy, cyclopropoxy, cyclopentyloxy, cyclohexyloxy, and the like.

The term “halogen” means fluorine, chlorine, bromine and iodine.

The term “heterocylyl” refers to a saturated or unsaturated monocyclic, bicyclic or tricyclic non-aromatic group including 1 to 5 heteroatoms selected from —O—, —S—, —S(O)—, —S(O)₂—, —N—, and —N(O)—. Examples of saturated monocyclic heterocyclic groups include morpholino, tetrahydrofuranyl, pyrrolidinyl, piperidinyl, tetrahydrothienyl, thiomorpholino, tetrahydropyranyl, butyrolactonyl, caprolactonyl, caprolactamyl, succinimidyl, and the like. Examples of unsaturated monocyclic heterocyclic groups include 2,3-dihydropyran, 2,3-dihydropyrrolidyl, 1,2-dihydropyridine, maleimidiyl, and the like. A bicyclic heterocyclyl radical includes fused bicyclic groups, bridged bicyclic groups, and spiro bicyclic groups.

The term “aryloxy,” alone or in combination, refers to an aryl ether radical, where “aryl” is as defined above. Examples include, but are not limited to, phenoxy and naphthyloxy. The term “heteroaryloxy” refers to a heteroaryl ether radical, where “heteroaryl” is as defined above. Examples include, but are not limited to, pyridyloxy, pyrrolyloxy, furyloxy, and thienyloxy.

The term “alkylthio,” alone or in combination, refers to an alkyl thioether radical, or cycloalkyl thioether radical, where the terms “alkyl” and “cycloalkyl” are as defined above. Examples of suitable alkyl thioether radicals include, but are not limited to, methylthio, ethylthio, n-propylthio, iso-propylthio, n-butylthio, iso-butylthio, sec-butylthio, tert-butylthio, cyclopropylthio, cyclopentylthio, cyclohexylthio, and the like.

The term “arylthio,” alone or in combination, refers to an aryl thioether radical, where “aryl” is as defined above. Examples include, but are not limited to, phenylthio and naphthylthio. The term “heteroarylthio” refers to a heteroaryl thioether radical, where “heteroaryl” is as defined above. Examples include, but are not limited to, pyridylthio, pyrrolylthio, furylthio, and thienylthio.

The term “arylene” refers to a carbocyclic aryl diradical, such as phenylene or naphthylene. The term “heteroarylene” refers to a heterocyclic aromatic diradical. Examples include but are not limited to pyridinylene, furylene, pyrimidinylene, and thienylene.

The compounds of formula (I) can be used for treating or preventing a disorder in which the blocking of purine receptors, particularly adenosine receptors and more particularly adenosine A_{2A} receptors, may be beneficial. The compounds can be administered to a subject in need of such treatment. For example, an effective dose of a compound of formula (I) or a pharmaceutically acceptable salt or prodrug thereof can be administered to a subject. The disorder may be caused by the hyperfunctioning of the purine receptors.

Disorders of particular interest include those in which the blocking of purine receptors, particularly adenosine receptors and more particularly adenosine A_{2A} receptors, may be beneficial. These include movement disorders such as Parkinson's disease, drug-induced Parkinsonism, post-encephalitic Parkinsonism, Parkinsonism induced by poisoning (for example MIP, manganese, carbon monoxide) and post-traumatic Parkinson's disease (punch-drunk syndrome).

Other movement disorders in which the blocking of purine receptors, may be of benefit include progressive supernuclear palsy, Huntingtons disease, multiple system atrophy, corticobasal degeneration, Wilsons disease, Hallerrorden-Spatz disease, progressive pallidal atrophy, Dopa-responsive dystonia-Parkinsonism, spasticity or other disorders of the basal ganglia which result in abnormal movement or posture. The present invention may also be effective in treating Parkinson's with on-off phenomena; Parkinson's with freezing (end of dose deterioration); and Parkinson's with prominent dyskinesias.

The compounds of formula (I) may be used or administered in combination with one or more additional drugs useful in the treatment of movement disorders, such as L-DOPA or a dopamine agonist, the components being in the same formulation or in separate formulations for administration simultaneously or sequentially.

Other disorders in which the blocking of purine receptors, particularly adenosine receptors and more particularly adenosine A_{2A} receptors may be beneficial include acute and chronic pain; for example neuropathic pain, cancer pain, trigeminal neuralgia, migraine and other conditions associated with cephalic pain, primary and secondary hyperalgesia, inflammatory pain, nociceptive pain, tabes dorsalis, phantom limb pain, spinal cord injury pain, central pain, post-herpetic pain and HIV pain; affective disorders including mood disorders such as bipolar disorder, seasonal affective disorder, depression, manic depression, atypical depression and monodepressive disease; central and peripheral nervous system degenerative disorders including corticobasal degeneration, demyelinating disease (multiple sclerosis, disseminated sclerosis), Friedrich's ataxia, motoneuron disease (amyotrophic lateral sclerosis, progressive bulbar atrophy), multiple system atrophy, myelopathy, radiculopathy, peripheral neuropathy (diabetic neuropathy, tabes dorsalis, drug induced neuropathy, vitamin deficiency), systemic lupus erythamatosis, granulomatous disease, olivo-ponto-cerebellar atrophy, progressive pallidal atrophy, progressive supranuclear palsy, spasticity; schizophrenia and related psychoses; cognitive disorders including dementia, Alzheimer's Disease, Frontotemporal dementia, multi-infarct dementia, AIDS dementia, dementia associated with Huntington's Disease, Lewy body dementia, senile dementia, age-related memory impairment, cognitive impairment associated with dementia, Korsakoff syndrome, dementia pugilans; attention disorders such as attention-deficit hyperactivity disorder (ADHD), attention deficit disorder, minimal brain dysfunction, brain-injured child syndrome, hyperkinetic reaction childhood, and hyperactive child syndrome; central nervous system injury including traumatic brain injury, neurosurgery (surgical trauma), neuroprotection for head injury, raised intracranial pressure, cerebral edema, hydrocephalus, spinal cord injury; cerebral ischemia including transient ischemic attack, stroke (thrombotic stroke, ischemic stroke, embolic stroke, hemorrhagic stroke, lacunar stroke) subarachnoid hemorrhage, cerebral vasospasm, neuroprotection for stroke, peri-natal asphyxia, drowning, cardiac arrest, subdural hematoma; myocardial ischemia; muscle ischemia; sleep disorders such as hypersomnia and narcolepsy; eye disorders such as retinal ischemia-reperfusion injury and diabetic neuropathy; cardiovascular disorders such as claudication and hypotension; and diabetes and its complications.

Compounds of formula (I) may be prepared according to conventional synthetic methods. For example compounds of formula (I) where R¹ is NH₂ may be synthesized by methods such as those illustrated in Reaction Scheme 1.

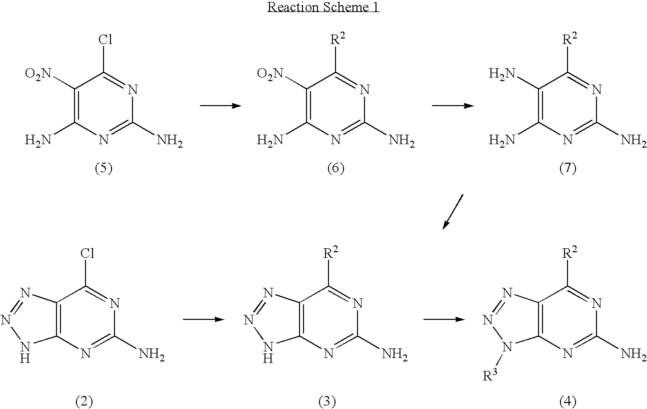

Compounds of formula (4) may be prepared from compounds of formula (3) by standard methods such as reaction with an appropriate alkyl halide, or substituted alkyl halide (e.g., an arylalkyl halide) in the presence of a suitable base such as sodium hydride.

Compounds of formula (4) where R³ is —C(O)N(R⁴)—Ar^{a}-N(R^{a})SO₃—R^{b} can be prepared from compounds of formula (4) where R³ is —COCl by standard methods such as direct reaction with an appropriate amine or hydrazine. In some cases, the compound of formula (4) includes R³ of the formula —C(O)N(R⁴)—Ar^{a}-NO₂. Reaction of such a compound with a dithionite salt (e.g., sodium dithionite, Na₂S₂O₄) can produce a compound of formula (I) where R³ has the formula —C(O)N(R⁴)—Ar^{a}-N(R^{a})SO₃—R^{b}.

Compounds of formula (3) may be prepared from the known chloro compound of formula (2) by standard methods such as aryl or heteroaryl coupling reactions. Suitable aryl or heteroaryl coupling reactions would include reaction with an appropriate aryl- or heteroaryl-boronic acid derivative, an aryl- or heteroaryl-trialkylstannane derivative or an aryl- or heteroaryl-zinc halide derivative in the presence of a suitable catalyst such as a palladium complex.

Compounds of formula (3) may also be prepared from compounds of formula (7) by standard methods such as treatment with isoamyl nitrite or sodium nitrite. Compounds of formula (7) are either known in the literature or can be prepared from compounds of formula (6) by standard methods such as reduction with hydrogen in the presence of a suitable catalyst such as Pd. Compounds of formula (6) are either known in the literature or can be prepared from the known compound of formula (5) by standard methods such as aryl or heteroaryl coupling reactions as described above.

Compounds of formula (I) where R¹ is —NR⁵R⁶ may be prepared from compounds of formula (4) by standard methods such as reductive amination with an appropriate aldehyde or ketone, or by treatment with an appropriate alkyl halide in the presence of a suitable base.

Compounds of formula (I) where R¹ is —NR^{a}CONR⁵R⁶, where R^{a} is H, may be prepared from compounds of formula (4) by standard methods such as treatment with an appropriate isocyanate R⁵NCO or R⁶NCO) or carbamoyl chloride R⁵R⁶NC(O)Cl). Compounds of formula (I) where R¹ is NR^{a}CONR⁵R⁶, where R^{a} is alkyl, may be prepared as described above having first performed an additional alkylation step as described above.

Compounds of formula (I) where R¹ is —NR^{a}COR⁴, —NR^{a}CO₂R⁴ or —NR^{a}SO₂R⁴, where R^{a} is H, may be prepared from compounds of formula (4) by standard methods such as treatment with an appropriate acid chloride (R⁵COCl), chloroformate (ClCO₂R⁴) or sulfonyl chloride (R⁴SO₂Cl) in the presence of a suitable base. Compounds of formula (I) where R¹ is —NR⁴COR⁴, —NR^{a}CO₂R⁴ or —NR^{a}SO₂R⁴, where R^{a} is alkyl may be prepared as described above having first performed an additional alkylation step as described above.

Compounds of formula (I) where R₁ is —NH₂ may also be synthesized by standard methods such as those illustrated in Reaction Scheme 2.

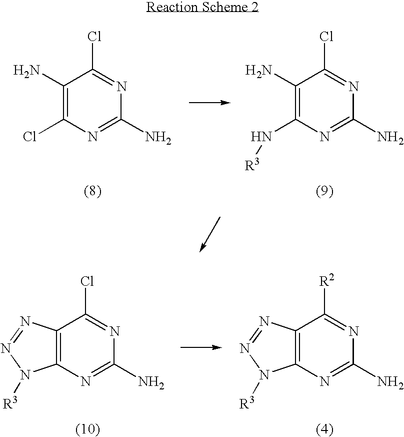

Compounds of formula (4) may be prepared from compounds of formula (10) by standard methods such as aryl or heteroaryl coupling reactions as described above. Compounds of formula (10) where R³ is arylalkyl are can be prepared by methods analogous to those described in the literature. For example compounds of formula (10) where R³ is arylalkyl may be prepared from compounds of formula (9) where R³ is arylalkyl by standard methods such as treatment with isoamyl nitrite or sodium nitrite. Compounds of formula (9) where R³ is arylalkyl can be prepared by methods described in the literature such as the treatment of the compound of formula (8) with an appropriate amine in a suitable solvent at elevated temperature.

Compounds of formula (10) can also be prepared by a modified version of Reaction Scheme 2, in which the 5-amino group of compound (8) is protected, as shown in Reaction Scheme 2A.

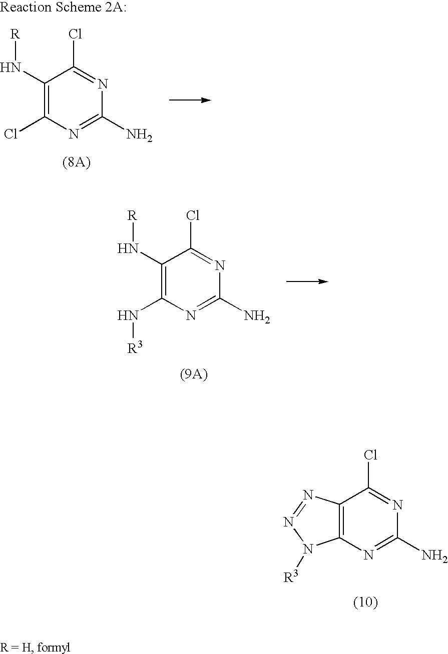

Compounds of formula (10) can be prepared from compounds of formula (9A) by standard methods such as treatment with isoamyl nitrite or sodium nitrite. Compounds of formula (9A) where R³ is arylalkyl can be prepared by methods such as the treatment of the compound of formula (8A) with an appropriate amine in a suitable solvent at elevated temperature.

Compounds of formula (I) where R¹ is —NH₂ may also be synthesized by standard methods such as those illustrated in Reaction Scheme 3.

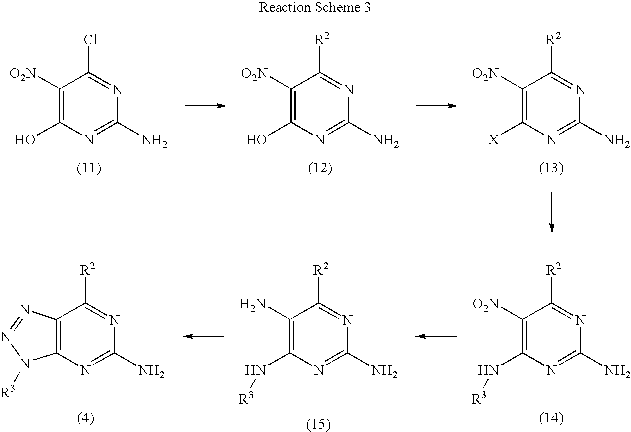

Compounds of formula (4) where R³ is arylalkyl can be prepared from compounds of formula (15) where R³ is arylalkyl by standard methods such as treatment with isoamyl nitrite. Compounds of formula (15) where R³ is arylalkyl may be prepared from compounds of formula (14) where R³ is arylalkyl by standard methods such as reduction with hydrogen in the presence of a suitable catalyst such as Pd. Compounds of formula (14) where R³ is arylalkyl may be prepared from compounds of formula (13), where X is a suitable leaving group such as a tosylate or triflate group, by standard methods such as treatment with a suitable amine in the presence of a suitable base such as triethylamine. Compounds of formula (13) where X is a suitable leaving group are either known in the literature or may be prepared from compounds of formula (12) by standard methods such as treatment with tosyl chloride or triflic anhydride in the presence of a suitable base such as triethylamine or 2,6-dimethylpyridine. Compounds of formula (12) are either known in the literature or may be prepared from the known compound of formula (11) by standard methods such as aryl or heteroaryl coupling reactions as described above.

Other compounds of formula (I) may be prepared by standard methods such as those illustrated in Reaction Scheme 4.

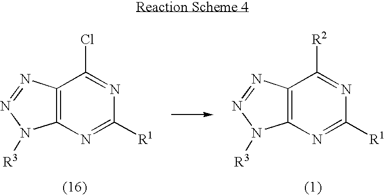

Compounds of formula (I) can be prepared from compounds of formula (16) by standard methods such as aryl or heteroaryl coupling reactions as described above. Compounds of formula (I) where R¹ is alkoxy, aryloxy, alkylthio, arylthio, —CN or —NR⁵R⁶ can be prepared from compounds of formula (I) where R¹ is halogen by standard methods such as nucleophilic displacement using an appropriate nucleophilic reagent such as an alcohol, thiol, cyanide or amine (NHR⁵R⁶) in the presence of a suitable base if required. Compounds of formula (1) where R¹ is halogen may be prepared from compounds of formula (16) where R¹ is halogen as described above. Compounds of formula (16) where R¹ is halogen are either known in the literature or may be prepared by methods analogous to those described in the literature.

Compounds of formula (I) where R¹ is —NR^{a}CONR⁵R⁶, —NR^{a}COR⁴, —NR^{a}CO₂R⁴ or —NR^{a}SO₂R⁴, where R^{a} is alkyl or aryl, may be prepared from compounds of formula (I) where R¹ is —NR⁵R⁶, where R⁵ is —H and R⁶ is alkyl or aryl, by the methods described above.

In certain cases it may be advantageous to prepare a compound of where R³ is selected to perform the function of a protecting group, for example a suitable protecting group would be a benzyl group or substituted benzyl group such as a 3,4-dimethoxybenzyl group. Compounds of this nature may prepared as described above and the protecting group R³ may be removed by standard methods such as treatment with, for example, TFA to give a compound where R³ is —H, Compounds of formula (I) where R³ is —H may then be used to prepare other compounds of formula (I), where R³ is as previously defined, by the methods described above.

In particular, compound of formula (I) can be prepared according to Reaction Scheme 5.

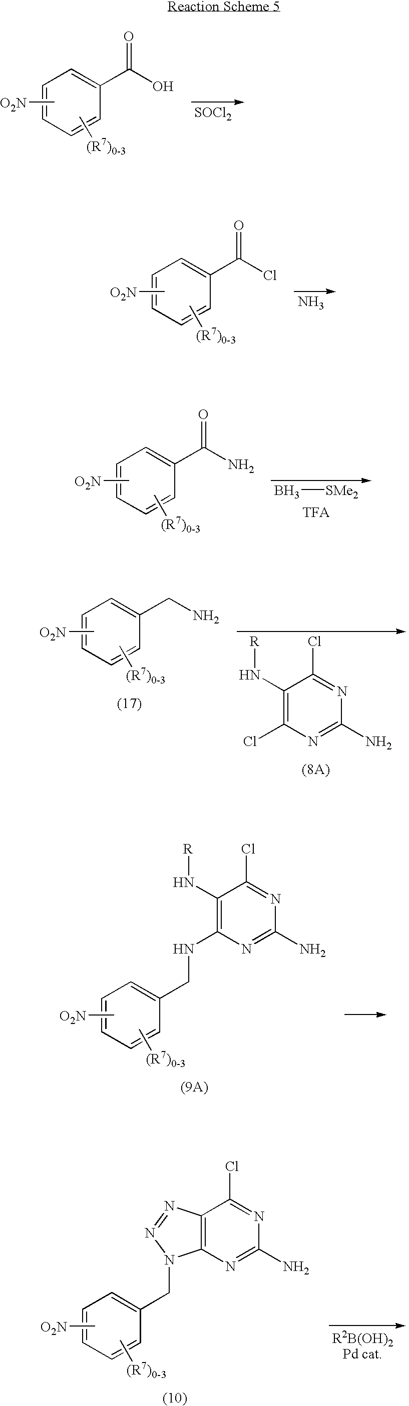

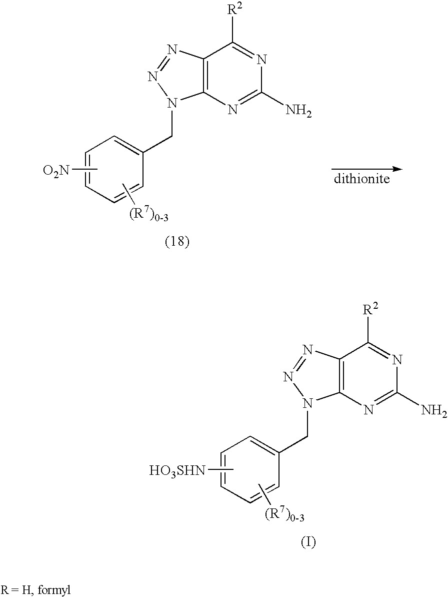

A (nitrophenylmethyl)amine (17) can be prepared from the corresponding nitrobenzoic acid as shown. Reaction of the (nitrophenylmethyl)amine (17) with compound (8A) yields a compound of formula (9A), which is converted to a compound of formula (10) by treatment with sulfuric acid and sodium nitrite. A metal catalyzed aryl or heteroaryl coupling reaction affords a nitro compound of formula (18). Reaction of (18) with sodium dithionite produces the compound of formula (I).

Alternatively, a compound of formula (I) is prepared from a compound of formula (18) according to reaction scheme 6:

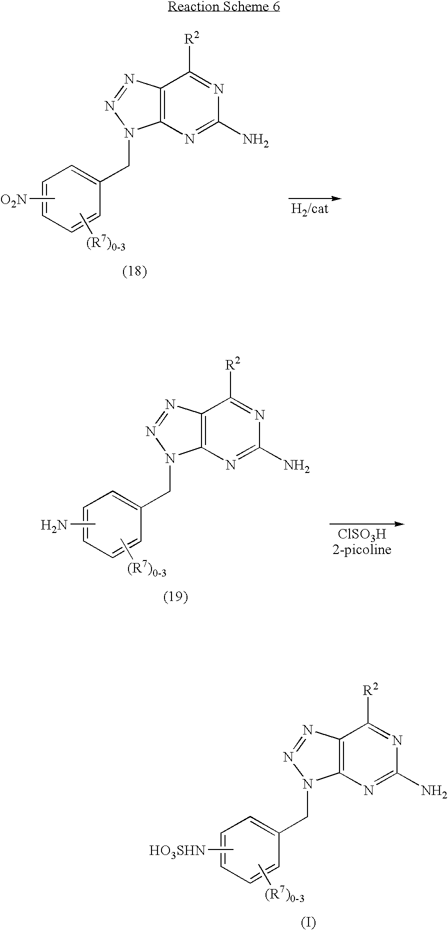

Instead of reacting a compound of formula (18) with sodium dithionite, (18) is reduced to aniline form by hydrogenation (19). The compound of formula (19) is then reacted with chlorosulfonic acid in the presence of 2-picoline to afford a compound of formula (I).

Compounds of formula (I) can be used in the form of pharmaceutically acceptable salts derived from inorganic or organic acids and bases. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth. Also, the basic nitrogen-containing groups can be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

The compound may be formulated into pharmaceutical compositions that may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutical compositions can include a compound of formula (I), or pharmaceutically acceptable derivatives thereof, together with any pharmaceutically acceptable carrier. The term “carrier” as used herein includes acceptable adjuvants and vehicles. Pharmaceutically acceptable carriers that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions may be in the form of a sterile injectable preparation, for example a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as do natural pharmaceutically-acceptable oils, such as olive oil or castor oil, especially in their polyoxyethylated versions. These oil solutions or suspensions may also contain a long-chain alcohol diluent or dispersant.

The pharmaceutical compositions can be orally administered in any orally acceptable dosage form including, but not limited to, capsules, tablets, aqueous suspensions or solutions.

In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions may be administered in the form of suppositories for rectal administration. These can be prepared by mixing the agent with a suitable non-irritating excipient which is solid at room temperature but liquid at the rectal temperature and therefore will melt in the rectum to release the drug. Such materials include cocoa butter, beeswax and polyethylene glycols.

The pharmaceutical compositions may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions may also be administered by nasal aerosol or inhalation through the use of a nebulizer, a dry powder inhaler or a metered dose inhaler. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of active ingredient that may be combined with the carrier materials to produce a single dosage form will vary depending upon the host treated, and the particular mode of administration. It should be understood, however, that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredient may also depend upon the therapeutic or prophylactic agent, if any, with which the ingredient is co-administered.

A pharmaceutical composition can include an effective amount of a compound of formula (I). An effective amount is defined as the amount which is required to confer a therapeutic effect on the treated patient, and will depend on a variety of factors, such as the nature of the inhibitor, the size of the patient, the goal of the treatment, the nature of the pathology to be treated, the specific pharmaceutical composition used, and the judgment of the treating physician. For reference, see Freireich et al., Cancer Chemother. Rep. 1966, 50, 219 and Scientific Tables, Geigy Pharmaceuticals, Ardley, N.Y., 1970, 537. Dosage levels of between about 0.001 and about 100 mg/kg body weight per day, preferably between about 0.1 and about 10 mg/kg body weight per day of the active ingredient compound are useful.

The following examples are for the purpose of illustration only and are not intended to be limiting.

### EXAMPLES

Compounds of formula I were prepared according to the scheme below, and as described in greater detail below.

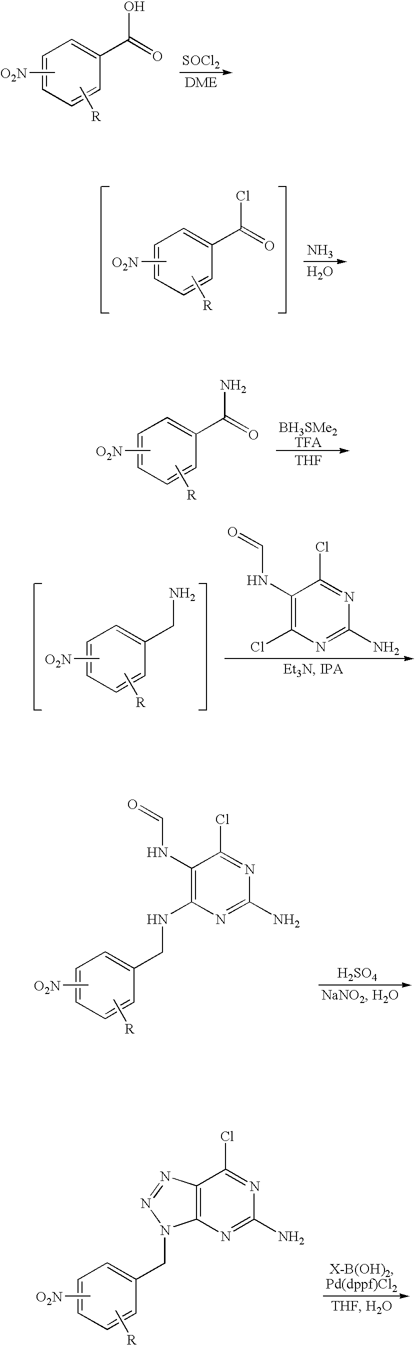

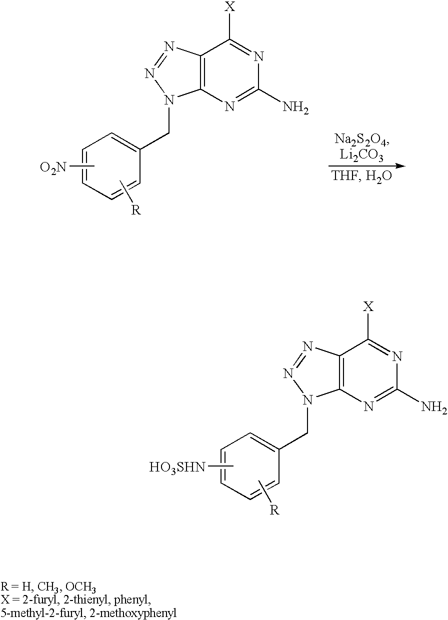

### 3-Methyl-4-nitro-benzamide

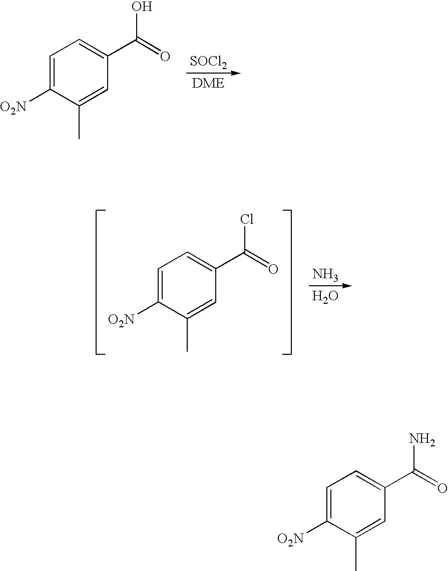

A stirred mixture of 3-methyl-4-nitro-benzoic acid (1.114 kg, 6.09 mol), N,N-dimethylformamide (4.6 mL, 0.06 mol), thionyl chloride (0.49 L, 6.65 mol) and 1,2-dimethoxyethane (2.20 L) was heated at 69-72° C. for 2 hours. The reaction mixture was then cooled to room temperature and was added into a stirred solution of ammonia hydroxide (3.30 L, 14.8 M in water) in water (5.50 L) at 10-15° C. The solid was collected by filtration and washed with water to afford 3-methyl-4-nitro-benzamide (1.073 kg, 97.8%) as a light yellow solid. MS m/e: 181 (M+H⁺).

Analogous conditions were used to prepare 4-methyl-3-nitrobenzamide, 3-methoxy-4-nitrobenzamide, and 2-methoxy-4-nitrobenzamide.

### N-[2-Amino-4-chloro-6-(3-methyl-4-nitro-benzylamino)-pyrimidin-5-yl]-formamide

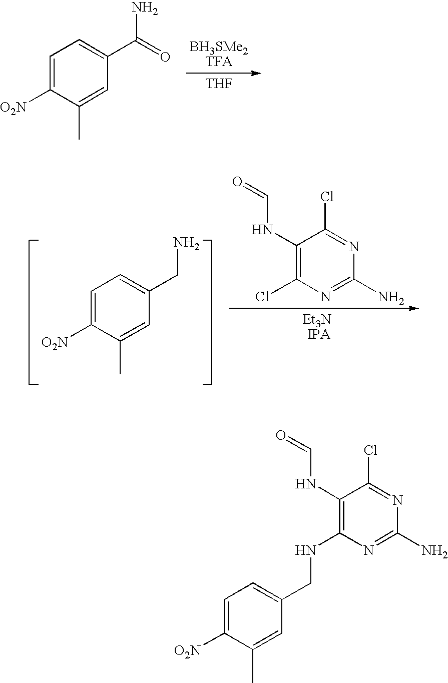

To a stirred slurry of 3-methyl-4-nitro-benzamide (104.0 g, 0.58 mol) in tetrahydrofuran (500 mL) were added trifluoroacetic acid (89.3 mL, 1.16 mol) and borane-dimethylsulfide (232 mL, 2.32 mol) at 60 to 65° C. and stirring continued for 2 hours at the same temperature. The reaction mixture was then cooled to 40° C. and isopropyl alcohol (1.5 L), triethylamine (168 mL, 1.21 mol) and N-(2-Amino-4,6-dichloro-pyrimidin-5-yl)-formamide (100.0 g, 0.48 mol) were added. The resulting slurry was heated at 75° C. and stirring continued for 4-6 hours. The solid was collected by filtration and washed with isopropyl alcohol to afford N-[2-amino-4-chloro-6-(3-methyl-4-nitro-benzylamino)-pyrimidin-5-yl]-formamide (194.5 g, 72 wt %, 86.1%) as a light yellow crystalline solid. MS m/e: 337 (M+H⁺).

Analogous conditions were used to prepare N-[2-amino-4-chloro-6-(4-methyl-3-nitrobenzylamino)pyrimidin-5-yl]-formamide, N-[2-amino-4-chloro-6-(3-methoxy-4-nitrobenzylamino)pyrimidin-5-yl]-formamide, and N-[2-amino-4-chloro-6-(2-methoxy-4-nitrobenzylamino)pyrimidin-5-yl]-formamide.

### 2-Amino-4-chloro-6-(3-methyl-4-nitro-benzylamino)-pyrimidin-5-yl-amine

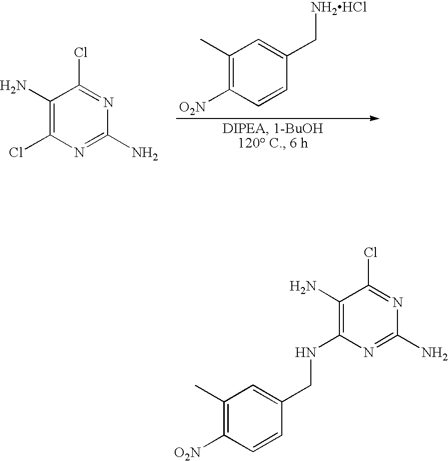

A 50 mL, rounded bottom flask was charged with 2,5-diamino-4,6-dichloropyrimidine (390 mg, 2.2 mmoles), and 3-methyl-4-nitrobenzylamine hydrochloride (500 mg, 2.5 mmoles). The vessel was then evacuated and flushed with nitrogen, then 1-butanol (8 mL) and diisopropylethylamine (0.86 mL, 4.9 mmoles) was added via syringe. The slurried material was then heated to 120° C. (reflux) over the course of a few minutes and held at that temperature for 6 hours. The reaction was complete and clean by HPLC. Treatment with sulfuric acid or TBME, afforded oils.

Use of IPA in place of 1-butanol, resulted in a slower reaction, due to the lower reflux temperature of IPA. This reaction took 12 hours and went nearly to completion with 6% of 5-diamino-4,6-dichloropyrimidine remaining. The product precipitated from the reaction mixture and was recovered by filtration. Yield: 0.3634 g, purity 95.9%, 54% yield

### 7-Chloro-3-(3-methyl-4-nitro-benzyl)-3H-[1,2,3]triazolo[4,5-d]pyrimidin-5-ylamine

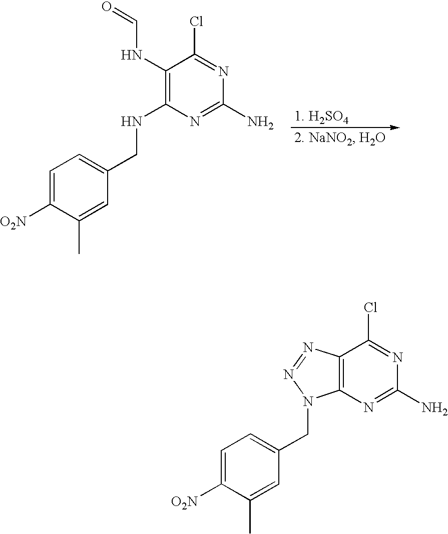

A stirred mixture of methanol (500 mL), sulfuric acid (23.6 mL, 423 mmol) and N-[2-amino-4-chloro-6-(3-methyl-4-nitro-benzylamino)-pyrimidin-5-yl]-formamide (50.0 g, 141 mmol) was heated at 60-70° C. for 1.5 hours with concomitant removal of formic acid methyl ester and methanol by distillation from the reaction flask. The reaction mixture was then cooled to 20° C. and was added water (200 mL) followed by the addition of sodium nitrite (21.0 mL, 160 mmol, 40 wt % in water) over 2 hours at 20° C. The solid was isolated by filtration and washed with water and 0.2 N ammonia hydroxide to afford 7-chloro-3-(3-methyl-4-nitro-benzyl)-3H-[1,2,3]triazolo[4,5-d]pyrimidin-5-ylamine (45.3 g, 98%) as a white crystalline solid. MS m/e: 320 (M+H⁺).

Analogous conditions were used to prepare 7-chloro-3-(4-methyl-3-nitro-benzyl)-3H-[1,2,3]triazolo[4,5-d]pyrimidin-5-ylamine, 7-chloro-3-(3-methoxy-4-nitro-benzyl)-3H-[1,2,3]triazolo[4,5-d]pyrimidin-5-ylamine, and 7-chloro-3-(2-methoxy-4-nitro-benzyl)-3H-[1,2,3]triazolo[4,5-d]pyrimidin-5-ylamine.

### 7-(furan-2-yl)-3-(3-methyl-4-nitrobenzyl)-3H-[1,2,3]triazolo[4,5-d]pyrimidin-5-amine

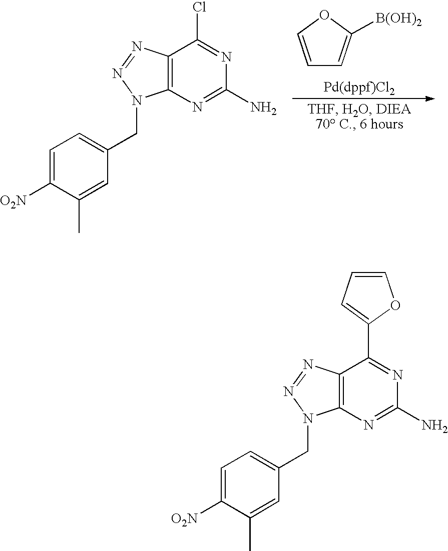

A 2 L, 3-neck rounded bottom flask, equipped with a mechanical stirrer, was charged with 7-chloro-3-(3-methyl-4-nitrobenzyl)-3H-[1,2,3]triazolo[4,5-d]pyrimidin-5-amine (50.0 g, 156.4 mmol), and Pd(dppf)Cl₂ (250 mg, 0.310 mmol). The vessel was then evacuated and flushed with nitrogen 3 times to remove oxygen. Next, water (175 mL) and THF (325 mL) was added via cannula, followed by diisopropylethylamine (81.7 mL, 469 mmol). The slurried material was then heated to 70° C. over the course of a half hour and held at that temperature for 30 minutes. A 200 mL Schlenk flask was charged 2-furylboronic acid (21.0 g, 188 mmoles). The flask was flushed with nitrogen and THF (75 mL) was added via a cannula. After all the boronic acid had dissolved, the solution was added to the 2 L reaction vessel with a cannula over the course of 20 minutes. The reaction temperature was maintained at 70° C. during the addition. The reaction was allowed to stir at 70° C. for an additional 2 hours, and then water (125 mL) was added all at once. The reaction was cooled to 25° C. The final product, off-white to pale yellow crystals, was collected by filtration. The filter cake was washed with methanol (200 mL in two parts) to remove any colored impurities. The 7-(furan-2-yl)-3-(3-methyl-4-nitrobenzyl)-3H-[1,2,3]triazolo[4,5-d]pyrimidin-5-amine was dried in a desiccator at 100 microns vacuum to constant weight to obtain 49.3 g; purity 98.8 A %, 90% yield (uncorrected for purities). MS m/e: 352.13 (M+H⁺).

Analogous conditions were used to prepare 7-(furan-2-yl)-3-(4-nitrobenzyl)-3H-[1,2,3]triazolo[4,5-d]pyrimidin-5-amine, 3-(4-nitrobenzyl)-7-(phenyl)-3H-[1,2,3]triazolo[4,5-d]pyrimidin-5-amine, 3-(4-nitrobenzyl)-7-(thiophen-2-yl)-3H-[1,2,3]triazolo[4,5-d]pyrimidin-5-amine, 7-(2-methoxyphenyl)-3-(4-nitrobenzyl)-3H-[1,2,3]triazolo[4,5-d]pyrimidin-5-amine, 7-(furan-2-yl)-3-(3-nitrobenzyl)-3H-[1,2,3]triazolo[4,5-d]pyrimidin-5-amine, 3-(3-nitrobenzyl)-7-(thiophen-2-yl)-3H-[1,2,3]triazolo[4,5-d]pyrimidin-5-amine, 7-(2-methoxyphenyl)-3-(3-nitrobenzyl)-3H-[1,2,3]triazolo[4,5-d]pyrimidin-5-amine, 3-(3-nitrobenzyl)-7-phenyl-3H-[1,2,3]triazolo[4,5-d]pyrimidin-5-amine, 7-(2-methoxyphenyl)-3-(3-methyl-4-nitrobenzyl)-3H-[1,2,3]triazolo[4,5-d]pyrimidin-5-amine, 3-(3-methyl-4-nitrobenzyl)-7-(thiophen-2-yl)-3H-[1,2,3]triazolo[4,5-d]pyrimidin-5-amine, 3-(4-methyl-3-nitrobenzyl)-7-(thiophen-2-yl)-3H-[1,2,3]triazolo[4,5-d]pyrimidin-5-amine, 7-(furan-2-yl)-3-(4-methyl-3-nitrobenzyl)-3H-[1,2,3]triazolo[4,5-d]pyrimidin-5-amine, 3-(4-methyl-3-nitrobenzyl)-7-phenyl-3H-[1,2,3]triazolo[4,5-d]pyrimidin-5-amine, 7-(2-methoxyphenyl)-3-(4-methyl-3-nitrobenzyl)-3H-[1,2,3]triazolo[4,5-d]pyrimidin-5-amine, 3-(3-methoxy-4-nitrobenzyl)-7-(2-methoxyphenyl)-3H-[1,2,3]triazolo[4,5-d]pyrimidin-5-amine, 3-(3-methoxy-4-nitrobenzyl)-7-phenyl-3H-[1,2,3]triazolo[4,5-d]pyrimidin-5-amine, 3-(3-methoxy-4-nitrobenzyl)-7-(thiophen-2-yl)-3H-[1,2,3]triazolo[4,5-d]pyrimidin-5-amine, 7-(furan-2-yl)-3-(3-methoxy-4-nitrobenzyl)-3H-[1,2,3]triazolo[4,5-d]pyrimidin-5-amine, 3-(2-methoxy-4-nitrobenzyl)-7-(thiophen-2-yl)-3H-[1,2,3]triazolo[4,5-d]pyrimidin-5-amine, 7-(furan-2-yl)-3-(2-methoxy-4-nitrobenzyl)-3H-[1,2,3]triazolo[4,5-d]pyrimidin-5-amine, 3-(2-methoxy-4-nitrobenzyl)-7-phenyl-3H-[1,2,3]triazolo[4,5-d]pyrimidin-5-amine, 3-(2-methoxy-4-nitrobenzyl)-7-(2-methoxyphenyl)-3H-[1,2,3]triazolo[4,5-d]pyrimidin-5-amine, 7-(5-methylfuran-2-yl)-3-(4-nitrobenzyl)-3H-[1,2,3]triazolo[4,5-d]pyrimidin-5-amine, 7-(5-methylfuran-2-yl)-3-(3-nitrobenzyl)-3H-[1,2,3]triazolo[4,5-d]pyrimidin-5-amine, 3-(4-methyl-3-nitrobenzyl)-7-(5-methylfuran-2-yl)-3H-[1,2,3]triazolo[4,5-d]pyrimidin-5-amine, 3-(3-methyl-4-nitrobenzyl)-7-(5-methylfuran-2-yl)-3H-[1,2,3]triazolo[4,5-d]pyrimidin-5-amine, 3-(3-methoxy-4-nitrobenzyl)-7-(5-methylfuran-2-yl)-3H-[1,2,3]triazolo[4,5-d]pyrimidin-5-amine, and 3-(2-methoxy-4-nitrobenzyl)-7-(5-methylfuran-2-yl)-3H-[1,2,3]triazolo[4,5-d]pyrimidin-5-amine.

### 4-((5-amino-7-(furan-2-yl)-3H-[1,2,3]triazolo[4,5-d]pyrimidin-3-yl)methyl)-2-methylphenylsulfamic acid

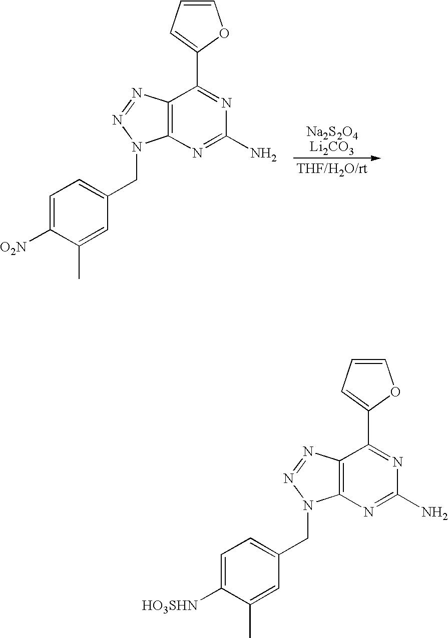

To a mixture of 7-(furan-2-yl)-3-(3-methyl-4-nitrobenzyl)-3H-[1,2,3]triazolo[4,5-d]pyrimidin-5-amine (5 g, 14.2 mmol), lithium carbonate (3.2 g, 43 mmol) and THF (70 mL) in a rounded bottom flask, was added slowly a solution of sodium dithionite (12.4 g, 60.5 mmol) in water (50 mL). The resulting yellowish slurry was stirred at ambient temperature for 2-16 hours until the nitro starting material was fully consumed. To the THF layer was added ⅓ volume of DMSO and the solution was purified by preparative HPLC. The isolated product fraction was collected and concentrated. The product was obtained as white fluffy solid via lyophilization

(1.01 g; 17.7% yield; 99 A % purity; MS m/e: 400.15 (M−H⁺); Exact MS: positive (m/e=402.0980) and negative (m/e=400.0838)).

The compounds presented in Table 1 were prepared in an analogous manner. Some compounds were partially hydrolyzed to the corresponding aniline derivative in the NMR solvent DSMO-d₆. This is denoted in Table 1 by NMR data in italics. Table 1 also presents results of in vitro testing of the compounds as inhibitors of adenosine A₁ and A_{2A} receptors. In Table 1, the measured Kᵢ values are represented by the following symbols: A, 100 nM or less; B, 100 nM to 1,000 nM; C, 1,000 nM to 10,000 nM; D, more than 10,000 nM, and the measured selectivity ratios (Kᵢ A_{2A}/Kᵢ A₁) are represented by the following symbols: E, less than 5; F, 5 to 10; G, 10 to 20; H, more than 20.

TABLE 1

| | | | | | ¹³C NMR | | | |
|---|---|---|---|---|---|---|---|---|
| | | | MS | ¹H NMR | (400 MHz, | A₁ | A_{2A} | |
| | | Yield | m/e: | (400 MHz, | DMSO-d₆) | Kᵢ, | Kᵢ, | |
| Structure | Name | % | M − H⁺ | DMSO-d₆) δ | δ | nM | nM | A_{2A}/A₁ |
| 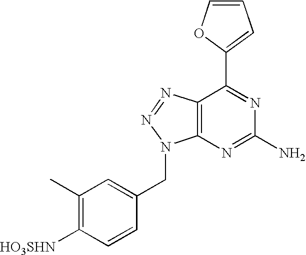 | 4-[(5-amino-7-(furan-2- yl)-3H- [1,2,3]triazolo[4,5- d]pyrimidin-3- yl)methyl]-2- methylphenylsulfamic acid | 17.7 | 400.15 | 8.12 (d, 1H), 7.91 (d, 1H), 7.42 (d, 1H), 7.34 (s, 2H), 7.03 (d, 1H), 6.95 (s, 1H), 6.86 (dd, 1H), 5.50 (s, 2H), 2.06 (s, 3H) | | B | A | H |
| 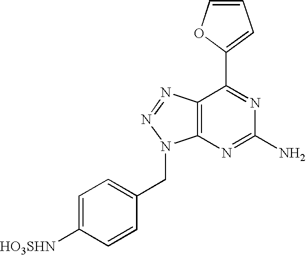 | 4-[(5-amino-7-(furan-2- yl)-3H- [1,2,3]triazolo[4,5- d]pyrimidin-3- yl)methyl]phenylsulfamic acid | 20 | 386.09 | 8.12 (d, 1H), 7.91 (d, 1H), 7.33 (s, 2H), 7.10 (d, 2H), 7.02 (d, 2H), 6.86 (dd, 1H), 5.51 (s, 2H) | 162.4, 151.6, 148.3, 147.3, 143.6, 128.7, 127.7, 125.7, 125.2, 118.7, 116.0, 113.0, 48.7 | C | A | H |
| 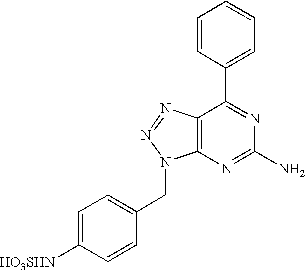 | 4-[(5-amino-7- (phenyl,2,3]triazolo[4,5- d]pyrimidin-3- yl)methyl]phenylsulfamic acid | 20 | 396.16 | 8.75 (m, 2H), 7.66 (m, 3H), 7.34 (s, 2H), 7.13 (d, 2H), 7.03 (d, 2H), 5.54 (s, 2H) | 163.5, 157.4, 152.3, 144.7, 134.1, 132.6, 128.8, 128.0, 127.5, 127.0, 125.1, 116.5, 47.0 | C | B | G |
| 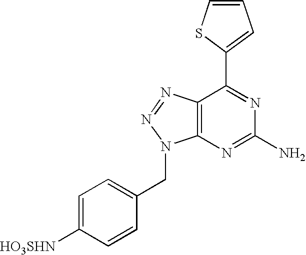 | 4-[(5-amino-7-(thiophen- 2-yl)-3H- [1,2,3]triazolo[4,5- d]pyrimidin-3- yl)methyl]phenylsulfamic acid | 9 | 402.11 | 8.68 (d, 1H), 7.98 (d, 1H), 7.38 (t, 1H), 7.27 (s, 2H), 7.11 (d, 2H), 7.02 (d, 2H), 5.51 (s, 2H) | 162.3, 152.5, 151.7, 143.6, 138.9, 133.7, 132.7, 129.1, 127.8, 126.4, 125.2, 116.1, 48.7 | C | B | G |
| 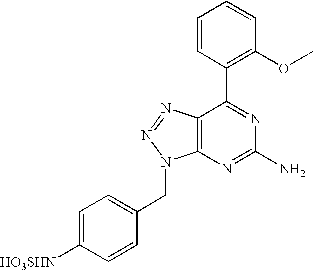 | 4-[(5-amino-7-(2- methoxyphenyl)-3H- [1,2,3]triazolo[4,5- d]pyrimidin-3- yl)methyl]phenylsulfamic acid | 27 | 426.14 | 7.53-7.50 (m, 2H), 7.31 (s, 2H), 7.23 (d, 1H), 7.14-7.10 (m, 3H), 7.03, (d, 2H), 5.49 (s, 2H), 3.78 (s, 3H) | 162.5, 160.0, 157.2, 150.8, 143.6, 131.6, 130.9, 129.5, 127.9, 125.4, 124.5, 120.2, 116.1, 112.0, 55.6, 48.7 | D | C | E |
| 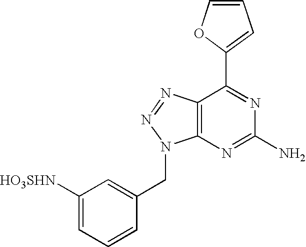 | 3-[(5-amino-7-(furan-2- yl)-3H- [1,2,3]triazolo[4,5- d]pyrimidin-3- yl)methyl]- phenylsulfamic acid | 34 | 386.11 | 8.12 (s, 1H), 7.29 (d, 1H), 7.36 (s, 2H), 7.06-6.99 (m, 3H), 6.86 (dd, 1H), 6.48 (d, 1H), 5.54 (s, 2H) | 162.5, 151.8, 148.3, 148.2, 147.3, 144.1, 135.6, 128.4, 125.6, 118.7, 116.7, 115.6, 114.8, 113.1, 49.0 | C | B | H |
| 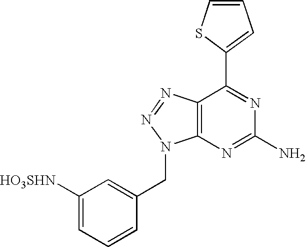 | 3-[(5-amino-7-(thiophen- 2-yl)-3H- [1,2,3]triazolo[4,5- d]pyrimidin-3- yl)methyl]- phenylsulfamic acid | 20 | 401.93 | 8.64 (dd, 1H), 7.93 (d, 1H), 7.32 (t, 1H), 7.24 (s, 2H), 6.99-6.91 (m, 3H), 6.41 (d, 1H), 5.48 (s, 2H) | 162.3, 152.5, 151.9, 144.1, 138.9, 135.6, 133.7, 132.8, 129.1, 128.4, 126.3, 116.6, 115.6, 114.8, 49.0 | C | B | G |
| 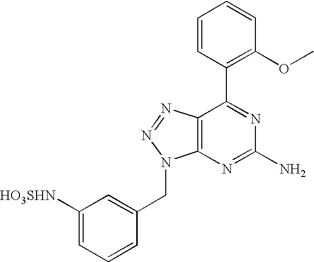 | 3-[(5-amino-7-(2- methoxyphenyl)-3H- [1,2,3]triazolo[4,5- d]pyrimidin-3- yl)methyl]- phenylsulfamic acid | 9 | 426.08 | 7.47-7.44 (m, 2H), 7.28 (s, 2H), 7.17 (d, 1H), 7.04 (t, 1H), 6.98-6.93 (m, 3H), 6.44 (d, 1H), 5.45 (s, 2H), 3.70 (s, 3H) | 162.6, 160.0, 157.3, 151.0, 144.1, 135.7, 131.6, 130.5, 129.4, 128.4, 124.5, 120.2, 116.9, 115.6, 115.0, 112.0, 55.6, 49.0 | C | C | E |
| 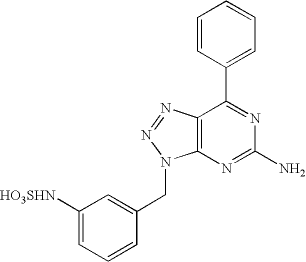 | 3-[(5-amino-7-phenyl- 3H-[1,2,3]triazolo[4,5- d]pyrimidin-3- yl)methyl]- phenylsulfamic acid | 20 | 396.00 | 8.77-8.74 (m, 2H), 7.66-7.63 (m, 3H), 7.37 (s, 2H), 7.06-6.98 (m, 3H), 6.49 (d, 2H), 5.57 (s, 2H) | 162.4, 157.5, 152.3, 144.1, 135.6, 134.5, 132.0, 129.3, 128.7, 128.4, 128.1, 116.6, 115.6, 114.8, 49.0 | C | A | G |
| 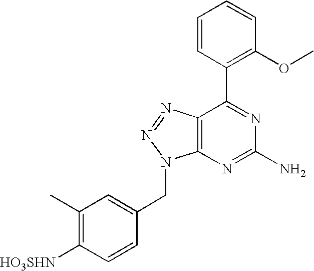 | 4-[(5-amino-7-(2- methoxyphenyl)-3H- [1,2,3]triazolo[4,5- d]pyrimidin-3- yl)methyl]-2- methylphenylsulfamic acid | 23 | 440.15 | 7.45-7.43 (m, 2H), 7.36 (d, 1H), 7.24 (s, 2H), 7.15 (d, 1H), 7.03 (t, 1H), 6.97 (d, 1H), 6.93 (s, 1H), 5.41 (s, 2H), 3.68 (s, 3H), 2.00 (s, 3H) | 162.5, 160.0, 157.2, 150.8, 141.1, 131.6, 130.5, 129.8, 129,5, 129.1, 125.6, 125.0, 124.5, 120.2, 117.4, 112.0, 55.6, 48.6, 17.6 | D | C | E |
| 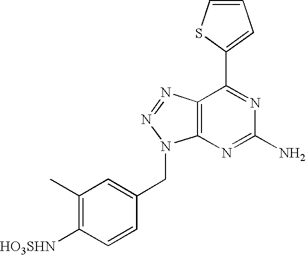 | 4-[(5-amino-7-(thiophen- 2-yl)-3H- [1,2,3]triazolo[4,5- d]pyrimidin-3- yl)methyl]-2- methylphenylsulfamic acid | 18 | 416.12 | 8.69 (d, 1H), 7.99 (d, 1H), 7.42-7.36 (m, 2H), 7.28 (s, 2H), 7.04 (d, 1H), 6.95 (s, 1H), 5.50 (s, 2H), 2.05 (s, 3H) | 162.3, 152.4, 151.6, 141.1, 138.9, 133.7, 132.7, 129.6, 129.1, 128.8, 126.4, 125.4, 124.5, 117.4, 48.7, 17.6 | B | A | H |
| 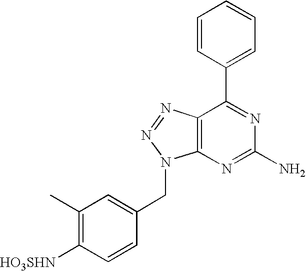 | 4-[(5-amino-7-phenyl- 3H-[1,2,3]triazolo[4,5- d]pyrimidin-3- yl)methyl]-2- methylphenylsulfamic acid | 20 | 410.17 | 8.76-8.73 (m, 2H), 7.65-7.63 (m, 3H), 7.44 (d, 1H), 7.35 (s, 2H), 7.05 (dd, 1H), 6.99 (d, 1H), 5.54 (s, 2H), 2.07 (s, 3H) | 162.3, 157.4, 152.1, 141.1, 134.5, 132.0, 129.3, 128.8, 128.2, 126.3, 126.1, 125.5, 124.3, 117.4, 48.7, 17.6 | B | A | H |
| 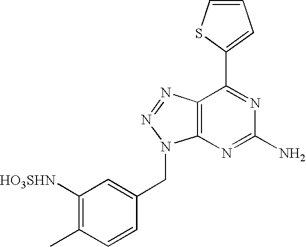 | 5-[(5-amino-7-(thiophen- 2-yl)-3H- [1,2,3]triazolo[4,5- d]pyrimidin-3- yl)methyl]-2- methylphenylsulfamic acid | 10 | 415.99 | 8.72 (d, 1H), 8.01 (d, 1H), 7.54 (d, 1H), 7.40 (dd, 1H), 7.35 (s, 2H), 6.94 (d, 1H), 6.42 (dd, 1H), 5.55 (s, 2H), 2.07 (s, 3H) | 162.3, 152.5, 151.8, 141.4, 138.9, 133.2, 132.8, 132.7, 129.7, 129.1, 126.3, 123.9, 117.5, 116.4, 49.0, 17.3 | C | A | H |
| 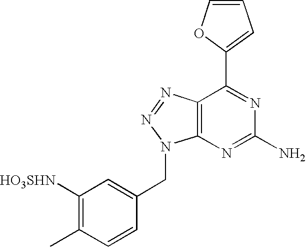 | 5-[(5-amino-7-(furan-2- yl)-3H- [1,2,3]triazolo[4,5- d]pyrimidin-3- yl)methyl]-2- methylphenylsulfamic acid | 24 | 399.89 | 8.05 (d, 1H), 7.85 (d, 1H), 7.46 (d, 1H), 7.31 (s, 2H), 6.86 (d, 1H), 6.80 (dd, 1H), 6.34 (dd, 1H), 5.47 (s, 2H), 1.99 (s, 3H) | 162.5, 151.7, 148.3, 148.2, 147.3, 141.4, 133.2, 129.7, 125.6, 123.9, 118.7, 117.5, 116.4, 113.0, 49.0, 17.3 | B | A | H |
| 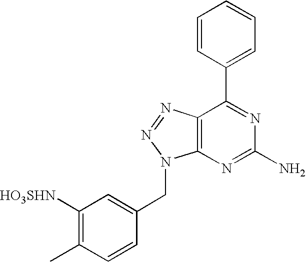 | 5-[(5-amino-7-phenyl- 3H-[1,2,3,]triazolo[4,5- d]pyrimidin-3- yl)methyl]-2- methylphenylsulfamic acid | 18 | 410.05 | 8.69-8.67 (m, 2H), 7.58-7.56 (m, 3H), 7.48 (d, 1H), 7.31 (s, 2H), 6.87 (d, 1H), 6.36 (dd, 1H), 5.50 (s, 2H), 1.99 (s, 3H) | 162.4, 157.4, 152.2, 141.4, 134.5, 134.1, 133.2, 130.1, 129.7, 129.3, 128.7, 123.9, 117.5, 116.4, 49.0, 17.3 | B | A | G |
| 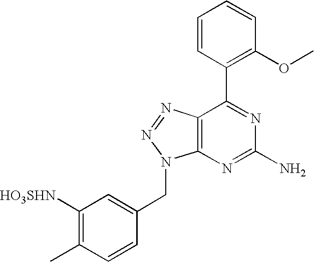 | 5-[(5-amino-7-(2- methoxyphenyl)-3H- [1,2,3]triazolo[4,5- d]pyrimidin-3- yl)methyl]-2- methylphenylsulfamic acid | 31 | 440.06 | 7.56-7.51 (m, 3H), 7.36 (s, 2H), 7.24 (d, 1H), 7.11 (t, 1H), 6.94 (d, 1H), 6.44 (d, 1H), 5.52 (s, 2H), 3.77 (s, 3H), 2.07 (s, 3H) | 162.6, 160.0, 157.3, 150.9, 141.4, 133.3, 131.6, 130.5, 129.7, 129.4, 124.5, 123.9, 120.2, 117.7, 116.6, 112.0, 55.6, 49.0, 17.4 | C | C | E |
| 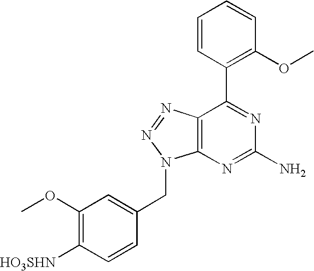 | 4-[(5-amino-7-(2- methoxyphenyl)-3H- [1,2,3]triazolo[4,5- d]pyrimidin-3- yl)methyl]-2- methoxyphenylsulfamic acid | 22 | 456.22 | 7.50-7.42 (m, 2H), 7.28 (d, 1H), 7.24 (s, 2H, collapsed after adding D₂O), 7.15 (d, 1H), 7.04 (t, 1H), 6.90 (d, 1H), 6.74 (dd, 1H), 6.21 (s, 1H, collapsed after adding D₂O), 5.45 (s, 2H), 3.69 (s, 3H), 3.31 (s, 3H) | 161.4, 158.9, 156.2, 149.8, 145.3, 131.0, 130.5, 129.4, 128.4, 125.4, 123.4, 119.1, 119.0, 114.7, 110.9, 108.8, 54.5, 54.4, 47.9 | D | D | E |
| 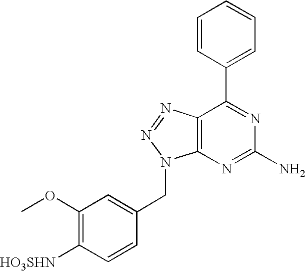 | 4-[(5-amino-7-phenyl- 3H-[1,2,3]triazolo[4,5- d]pyrimidin-3- yl)methyl]-2- methoxyphenylsulfamic acid | 26 | 426.14 | 8.75-8.73 (m, 2H), 7.65-7.63 (m, 3H), 7.37-7.35 (m, 3H), 6.97 (s, 1H), 6.80 (d, 1H), 5.58 (s, 2H), 3.76 (s, 3H) | 162.3, 157.5, 152.1, 146.4, 134.5, 132.1, 132.0, 129.3, 128.7, 128.2, 126.4, 120.0, 115.8, 109.7, 55.5, 49.0 | C | B | G |
| 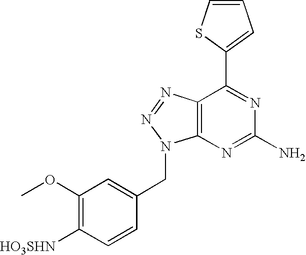 | 4-[(5-amino-7-(thiophen- 2-yl)-3H- [1,2,3]triazolo[4,5- d]pyrimidin-3- yl)methyl]-2- methoxyphenylsulfamic acid | 21 | 432.03 | 8.62 (d, 1H), 7.92 (d, 1H), 7.31-7.29 (m, 2H), 7.22 (s, 2H), 6.87 (s, 1H), 6.70 (d, 1H), 5.48 (s, 2H), 3.68 (s, 3H) | 162.3, 152.5, 151.7, 146.4, 138.9, 133.7, 132.7, 132.1, 129.1, 126.4, 126.3, 120.0, 115.8, 109.7, 55.5, 49.0 | C | B | H |
| 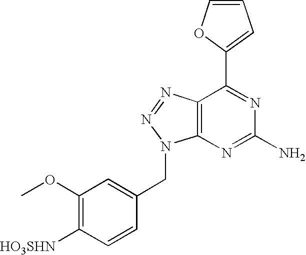 | 4-[(5-amino-7-(furan-2- yl)-3H- [1,2,3]triazolo[4,5- d]pyrimidin-3- yl)methyl]-2- methoxyphenylsulfamic acid | 32 | 416.05 | 8.17 (s, 1H), 7.95 (d, 1H), 7.39 (s + d, 3H, collapsed after adding D₂O), 6.99 (s, 1H), 6.90 (d, 1H), 6.83 (d, 1H), 6.35 (s, 1H, collapsed after adding D₂O), 5.59 (s, 2H), 3.80 (s, 3H) | 162.5, 151.6, 148.3, 148.2, 147.3, 146.4, 132.0, 126.4, 125.7, 119.9, 118.7, 115.8, 113.0, 109.7, 55.5, 48.9 | D | C | G |
| 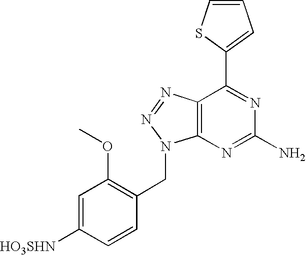 | 4-[(5-amino-7-(thiophen- 2-yl)-3H- [1,2,3]triazolo[4,5- d]pyrimidin-3- yl)methyl]-3- methoxyphenylsulfamic acid | 16 | 431.98 | 8.62 (dd, 1H), 7.92 (d, 1H), 7.30 (dd, 1H), 7.19 (s, 2H), 6.77 (d, 1H), 6.69 (d, 1H), 6.49 (dd, 1H), 5.40 (s, 2H), 3.62 (s, 3H) | 162.2, 156.7, 152.3, 151.9, 145.3, 139.0, 133.6, 132.6, 129.1, 128.5, 126.3, 113.0, 108.1, 99.5, 55.1, 43.9 | D | C | F |
| 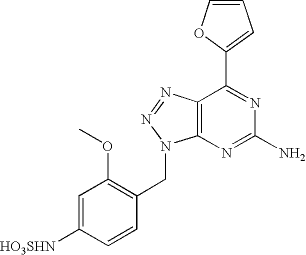 | 4-[(5-amino-7-(furan-2- yl)-3H- [1,2,3]triazolo[4,5- d]pyrimidin-3- yl)methyl]-3- methoxyphenylsulfamic acid | 7 | 416.03 | 8.04 (s, 1H), 7.83 (d, 1H), 7.22 (s, 2H), 6.79 (m, 2H), 6.68 (d, 1H), 6.49 (d, 1H), 5.39 (s, 2H), 3.62 (s, 3H) | 162.4, 156.7, 151.8, 148.4, 148.1, 147.2, 145.2, 128.5, 125.6, 118.6, 113.0, 108.1, 99.5, 55.1, 43.9 | D | B | G |
| 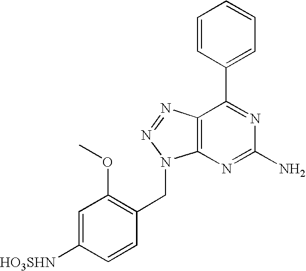 | 4-[(5-amino-7-phenyl- 3H-[1,2,3]triazolo[4,5- d]pyrimidin-3- yl)methyl]-3- methoxyphenylsulfamic acid | 15 | 426.08 | 8.79 (m, 2H), 7.95 (s, 1H, vanished after adding D₂O), 7.69-7.68 (m, 3H), 7.35 (s, 2H, collapsed after adding D₂O), 6.90 (d, 1H), 6.80 (d, 1H), 6.60 (dd, 1H), 5.55 (s, 2H), 3.75 (s, 3H) | 162.3, 157.3, 156.7, 152.3, 145.3, 134.9, 131.9, 129.2, 128.7, 128.5, 128.0, 113.0, 108.1, 99.5, 55.1, 43.9 | D | C | F |
| 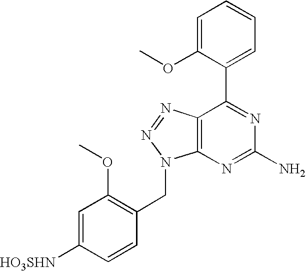 | 4-[(5-amino-7-(2- methoxyphenyl)-3H- [1,2,3]triazolo[4,5- d]pyrimidin-3- yl)methyl]-3- methoxyphenylsulfamic acid | 20 | 456.09 | 7.58-7.56 (m, 2H), 7.34 (s, 2H), 7.29 (d, 1H), 7.16 (t, 1H), 6.90 (d, 1H), 6.83 (d, 1H), 6.63 (dd, 1H), 5.51 (s, 2H), 3.81 (s, 3H), 3.76 (s, 3H) | 162.4, 159.8, 157.3, 156.8, 151.0, 145.3, 131.6, 130.3, 129.4, 128.7, 124.5, 120.2, 113.1, 112.0, 108.1, 99.5, 55.6, 55.1, 43.7 | D | C | E |
| 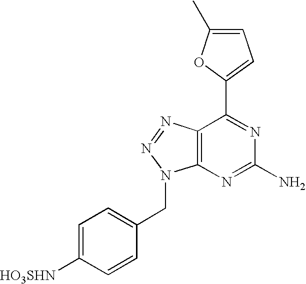 | 4-[(5-amino-7-(5- methylfuran-2-yl)-3H- [1,2,3]triazolo[4,5- d]pyrimidin-3- yl)methyl]phenylsulfamic acid | 21 | 399.98 | 7.79 (d, 1H), 7.21 (s, 2H), 7.02 (d, 2H), 6.94 (d, 2H), 6.43 (d, 1H), 5.41 (s, 2H), 2.38 (s, 3H) | 162.5, 157.0, 151.4, 147.9, 146.7, 143.6, 127.7, 125.4, 125.2, 120.7, 116.0, 109.8, 48.6, 13.6 | C | A | H |
| 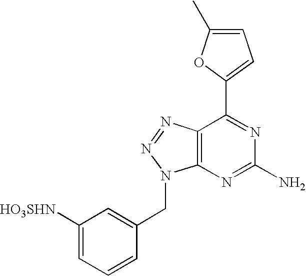 | 3-[(5-amino-7-(5- methylfuran-2-yl)-3H- [1,2,3]triazolo[4,5- d]pyrimidin-3- yl)methyl]phenylsulfamic acid | 16 | 399.99 | 7.81 (d, 1H), 7.26 (s, 2H), 6.98-6.90 (m, 3H), 6.44 (d, 1H), 6.40 (d, 1H), 5.45 (s, 2H), 2.38 (s, 3H) | 161.5, 155.9, 150.5, 146.9, 145.7, 143.0, 134.6, 127.3, 124.3, 119.6, 115.6, 114.5, 113.7, 108.7, 47.9, 12.5 | B | A | H |
| 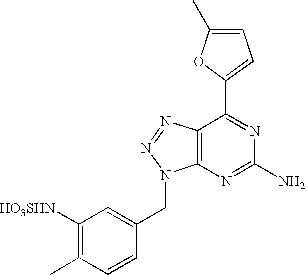 | 5-[(5-amino-7-(5- methylfuran-2-yl)-3H- [1,2,3]triazolo[4,5- d]pyrimidin-3- yl)methyl]-2- methylphenylsulfamic acid | 20 | 414.04 | 7.88 (d, 1H), 7.52 (s, 1H), 7.34 (s, 2H), 6.93 (d, 1H), 6.51 (d, 1H), 6.40 (d, 1H), 5.52 (s, 2H), 2.46 (s, 3H), 2.06 (s, 3H) | 162.5, 157.1, 151.5, 147.9, 146.8, 141.4, 133.3, 129.7, 125.4, 123.9, 120.7, 117.4, 116.4, 109.8, 49.0, 17.3, 13.6 | B | A | H |
| 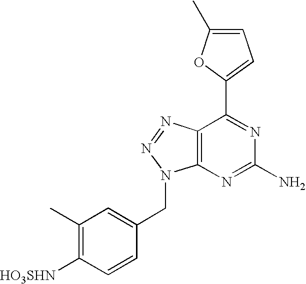 | 4-[(5-amino-7-(5- methylfuran-2-yl)-3H- [1,2,3]triazolo[4,5- d]pyrimidin-3- yl)methyl]-2- methylphenylsulfamic acid | 42 | 414.05 | 7.87 (d, 1H), 7.42 (d, 1H), 7.29 (s, 2H), 7.04 (dd, 1H), 6.95 (d, 1H), 6.50 (d, 1H), 5.48 (s, 2H), 2.45 (s, 3H), 2.05 (s, 3H) | 162.5, 157.0, 151.4, 147.9, 146.8, 141.1, 129.6, 128.8, 126.1, 125.4, 124.4, 120.7, 117.4, 109.8, 48.6, 17.6, 13.6 | B | A | H |
| 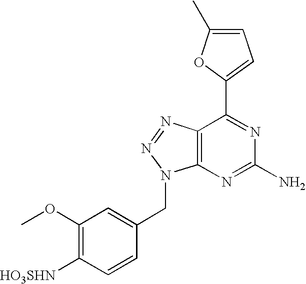 | 4-[(5-amino-7-(5- methylfuran-2-yl)-3H- [1,2,3]triazolo[4,5- d]pyrimidin-3- yl)methyl]-2- methoxyphenylsulfamic acid | 30 | 430.05 | 7.87 (d, 1H), 7.35 (d, 1H), 7.30 (s, 2H), 6.93 (d, 1H), 6.78 (dd, 1H), 6.50 (d, 1H), 5.53 (s, 2H), 3.75 (s, 3H), 2.45 (s, 3H) | 162.5, 157.0, 151.4, 147.9, 146.8, 146.4, 132.1, 126.4, 125.5, 120.7, 119.9, 115.8, 109.8, 109.7, 55.5, 48.9, 13.6 | B | A | H |
| 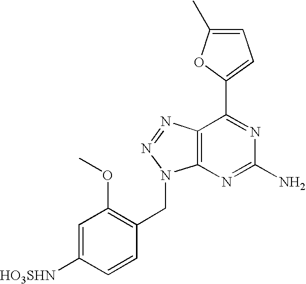 | 4-[(5-amino-7-(5- methylfuran-2-yl)-3H- [1,2,3]triazolo[4,5- d]pyrimidin-3- yl)methyl[-3- methoxyphenylsulfamic acid | 33 | 430.05 | 7.79 (d, 1H), 7.20 (s, 2H), 6.77 (d, 1H), 6.67 (d, 1H), 6.50 (dd, 1H), 6.43 (d, 1H), 5.38 (s, 2H), 3.62 (s, 3H), 2.38 (s, 3H) | 162.4, 156.9, 156.7, 151.6, 147.8, 146.8, 145.2, 128.5, 125.3, 120.6, 113.1, 109.8, 108.1, 99.5, 55.1, 43.8, 13.6 | C | B | H |

Adenosine Receptor Binding: Binding Affinities at hA₁ Receptors

The compounds were examined in an assay measuring in vitro binding to human adenosine A₁ receptors by determining the displacement of the adenosine A₁ receptor selective radioligand 8-Cyclopentyl-1,3-dipropylxanthine ([³H]DPCPX) using standard techniques. See, for example, Lohse M J, et al., (1987), 8-Cyclopentyl-1,3-dipropylxanthine (DPCPX)—a selective high affinity antagonist radioligand for A1 adenosine receptors. Naunyn Schmiedebergs Arch Pharmacol., 336(2):204-10, which is incorporated by reference in its entirety.

Frozen CHO-K1 cells (transfected with a human adenosine A₁ recepter expression vector) were homogenized in 130 mL of 50 mM Tris HCl buffer (pH 7.5) containing 10 mM MgCl₂, and 0.1 IU/mL adenosine deaminase per pellet using a Ultra-Turrax homogeniser. The resultant homogenate was kept for immediate use in the binding. Binding assays were performed in a total volume of 250 μL, containing [³H]-DPCPX (3.0 nM), membranes and additional drugs. Total binding was determined using drug dilution buffer (50 mM Tris-HCl pH:7.5, 10 mM MgCl₂, 5% DMSO). Non-specific binding was determined using 300 μM N6-cyclohexyladenosine (CHA). Following incubation for 90 minutes at 21° C., assays were terminated by rapid filtration with GF/B filters (presoaked in 0.1% (w/v) polyethylenimine) using a Canberra Packard filtermate 196, washed 3 times with ice-cold Tris-HCl (pH 7.4). Filters were left to dry overnight, and Microscint-0 scintillation fluid was then added to the filters. The filters were then left for at least 2 hours before the radioactivity was assessed using a Canberra Packard TopCount microplate scintillation counter.

To determine the free ligand concentration, three vials were counted with 25 μL of [³H]DPCPX containing 4 mL of Ultima-Gold MV scintillant on a Beckman LS6500 multi-purpose scintillation counter.

Data was analysed using a 4 parameter logistical equation and non-linear regression which yields affinity constants (pIC₅₀), and slope parameters:

$$E = NSB + \frac{Total - NSB}{1 + {( \frac{log [ {IC}_{50} ]}{log [ A ]} )}^{slope}}$$

where E is the quantity of binding and [A] is the competitor concentration. The K, is then determined using the Cheng-Prusoff equation:

$${K}_{i} = \frac{{IC}_{50}}{1 + ( \frac{[ L ]}{[ {K}_{D} ]} )}$$

Adenosine Receptor Binding: Binding Affinities at hA_{2A} Receptors

The compounds were examined in an assay measuring in vitro binding to human adenosine A_{2A} receptors by determining the displacement of the adenosine A_{2A} receptor selective radioligand 4-[2-[[6-Amino-9-(N-ethyl-β-D-ribofuranuronamidosyl)-9H-purin-2-yl]amino]ethyl]benzenepropanoic acid hydrochloride ([³H]CGS-21680) using standard techniques. See, for example, Jarvis et al., J Pharmacol Exp Ther., 251(3):888-93, which is incorporated by reference in its entirety.

Frozen HEK-293 cells were homogenized in 65 mL of 50 mM Tris HCl buffer (pH 7.5) containing 10 mM MgCl₂, and 0.1 IU/mL adenosine deaminase per pellet using a Ultra-Turrax homogenizer. The resultant homogenate was kept for immediate use in the binding assay.

Binding assays were performed in a total volume of 250 μL, containing [³H]-CGS21680 (20.0 nM), membranes and additional drugs. Total binding was determined using drug dilution buffer (50 mM Tris-HCl pH 7.5, 10 mM MgCl₂, 5% DMSO). Non-specific binding was determined using 300 μM CHA. Following incubation for 90 minutes at 21° C., assays were terminated by rapid filtration with GF/B filters (presoaked in 0.1% (w/v) polyethylenimine) using a Canberra Packard filtermate 196, washed 3 times with ice-cold Tris-HCl (pH 7.4). Filters were left to dry overnight, and Microscint-0 scintillation fluid was then added to the filters. The filters were then left for at least 2 hours before the radioactivity was assessed using a Canberra Packard TopCount microplate scintillation counter.

To determine the free ligand concentration, three vials were counted with 25 μL of [³H]CGS21680 containing 4 mL of Ultima-Gold MV scintillant on a Beckman LS6500 multi-purpose scintillation counter.

Data was analysed using a 4 parameter logistical equation and non-linear regression which yields affinity constants (pIC₅₀), and slope parameters:

$$E = NSB + \frac{Total - NSB}{1 + {( \frac{log [ {IC}_{50} ]}{log [ A ]} )}^{slope}}$$

where E is the quantity of binding and [A] is the competitor concentration. The Kᵢ is then determined using the Cheng-Prusoff equation:

$${K}_{i} = \frac{{IC}_{50}}{1 + ( \frac{[ L ]}{[ {K}_{D} ]} )}$$

### Evaluation of Potential Anti-Parkinsonian Activity In Vivo: Haloperidol-Induced Hypolocomotion Model

It has previously been demonstrated that adenosine antagonists, such as theophylline, can reverse the behavioral depressant effects of dopamine antagonists, such as haloperidol, in rodents (see, for example, Mandhane S, N. et al., Adenosine A₂ receptors modulate haloperidol-induced catalepsy in rats. Eur. J. Pharmacol. 1997, 328, 135-141, which is incorporated by reference in its entirety). This approach is also considered a valid method for screening drugs with potential antiparkinsonian effects. Thus, the ability of novel adenosine antagonists to block haloperidol-induced deficits in locomotor activity in mice can be used to assess both in vivo and potential antiparkinsonian efficacy.

Female TO mice (25-30 g) are used for all experiments. Animals are housed in groups of 8 (cage size-40 cm (width) by 40 cm (length) by 20 cm (height)) under 12 hour light/dark cycle (lights on 08:00), in a temperature (20±2° C.) and humidity (55±15%) controlled environment. Animals have free access to food and water, and are allowed at least 7 days to acclimatize after delivery before experimental use.

Liquid injectable haloperidol (e.g., 1 mL Serenance ampoules from Baker Norton, Harlow, Essex, each containing haloperidol BP 5 mg) are diluted to a final concentration of 0.02 mg/mL using saline. Test compounds are typically prepared as aqueous suspensions in 8% Tween. All compounds are administered intraperitoneally in a volume of 10 mL/kg.

1.5 hours before testing, mice are administered 0.2 mg/kg haloperidol, a dose that reduces baseline locomotor activity by at least 50%. Test substances are typically administered 5-60 minutes prior to testing. The animals are then placed individually into clean, clear polycarbonate cages (20 cm (width) by 40 cm (length) by 20 cm (height), with a flat perforated, Perspex lid). Horizontal locomotor activity is determined by placing the cages within a frame containing a 3 by 6 array of photocells linked to a computer, which tabulates beam breaks. Mice are left undisturbed to explore for 1 hour, and the number of beams breaks made during this period serves as a record of locomotor activity which is compared with data for control animals for statistically significant differences.

### Evaluation of Potential Anti-Parkinsonian Activity In Vivo: 6-OHDA Model

Parkinson's disease is a progressive neurodegenerative disorder characterized by symptoms of muscle rigidity, tremor, paucity of movement (hypokinesia), and postural instability. It has been established for some time that the primary deficit in PD is a loss of dopaminergic neurons in the substantia nigra which project to the striatum, and indeed a substantial proportion of striatal dopamine is lost (ca 80-85%) before symptoms are observed. The loss of striatal dopamine results in abnormal activity of the basal ganglia, a series of nuclei which regulate smooth and well coordinated movement (see, e.g., Blandini F. et al., Glutamate and Parkinson's Disease. Mol. Neurobiol. 1996, 12, 73-94, which is incorporated by reference in its entirety). The neurochemical deficits seen in Parkinson's disease can be reproduced by local injection of the dopaminergic neurotoxin 6-hydroxydopamine into brain regions containing either the cell bodies or axonal fibers of the nigrostriatal neurons.

By unilaterally lesioning the nigrostriatal pathway on only one-side of the brain, a behavioral asymmetry in movement inhibition is observed. Although unilaterally-lesioned animals are still mobile and capable of self maintenance, the remaining dopamine-sensitive neurons on the lesioned side become supersenstive to stimulation. This is demonstrated by the observation that following systemic administration of dopamine agonists, such as apomorphine, animals show a pronounced rotation in a direction contralateral to the side of lesioning. The ability of compounds to induce contralateral rotations in 6-OHDA lesioned rats has proven to be a sensitive model to predict drug efficacy in the treatment of Parkinson's Disease.

Male Sprague-Dawley rats, obtained from Charles River, are used for all experiments Animals are housed in groups of 5 under 12 hour light/dark cycle (lights on 08:00), in a temperature (20±2° C.) and humidity (55±5%) controlled environment. Animals have free access to food and water, and are allowed at least 7 days to acclimatize after delivery before experimental use.

Ascorbic acid, desipramine, 6-OHDA and apomorphine are obtained commercially. 6-OHDA is freshly prepared as a solution in 0.2% ascorbate at a concentration of 4 mg/mL prior to surgery. Desipramine is dissolved in warm saline, and administered in a volume of 1 mL/kg. Apomorphine is dissolved in 0.02% ascorbate and administered in a volume of 2 mL/kg. Test compounds are suspended in 8% Tween and injected in a volume of 2 mL/kg.

15 minutes prior to surgery, animals are given an intraperitoneal injection of the noradrenergic uptake inhibitor desipramine (25 mg/kg) to prevent damage to nondopamine neurons. Animals are then placed in an anaesthetic chamber. and anaesthetised using a mixture of oxygen and isoflurane. Once unconscious, the animals are transferred to a stereotaxic frame, where anaesthesia is maintained through a mask. The top of the animal's head is shaved and sterilized using an iodine solution. Once dry, a 2 cm long incision is made along the midline of the scalp and the skin retracted and clipped back to expose the skull. A small hole is then drilled through the skill above the injection site. In order to lesion the nigrostriatal pathway, the injection cannula is slowly lowered to position above the right medial forebrain bundle at −3.2 mm anterior posterior, −1.5 mm medial lateral from bregma, and to a depth of 7.2 mm below the duramater. 2 minutes after lowing the cannula, 2 VAL of 6-OHDA is infused at a rate of 0.5 μL/min over 4 minutes, yielding a final dose of 8 μg. The cannula is then left in place for a further 5 minutes to facilitate diffusion before being slowly withdrawn. The skin is then sutured shut using Ethicon W501 Mersilk, and the animal removed from the strereotaxic frame and returned to its homecage. The rats are allowed 2 weeks to recover from surgery before behavioral testing.

Rotational behavior is measured using an eight station rotameter system, such as one sold by Med Associates, San Diego, USA. Each station is comprised of a stainless steel bowl (45 cm diameter by 15 cm high) enclosed in a transparent Plexiglas cover running around the edge of the bowl, and extending to a height of 29 cm. To assess rotation, rats are placed in cloth jacket attached to a spring tether connected to optical rotameter positioned above the bowl, which assesses movement to the left or right either as partial)(45° or full)(360° rotations. All eight stations are interfaced to a computer that tabulated data.

To reduce stress during drug testing, rats are initially habituated to the apparatus for 15 minutes on four consecutive days. On the test day, rats are given an intraperitoneal injection of test compound 30 minutes prior to testing. Immediately prior to testing, animals are given a subcutaneous injection of a subthreshold dose of apomorphine, then placed in the harness and the number of rotations recorded for one hour. The total number of full contralatral rotations during the hour test period serves as an index of antiparkinsonian drug efficacy.

Other embodiments are within the scope of the following claims.

1. A compound of formula (I): 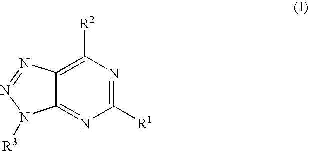
wherein
R¹ is selected from H, alkyl, aryl, heteroaryl, cycloalkyl, heterocyclyl, alkoxy, aryloxy, heteroaryloxy, alkylthio, arylthio, heteroarylthio, halogen, —CN, —NR⁵R⁶, —N(R^{a})C(O)R⁴, —N(R^{a})C(O)NR⁵R⁶, —N(R^{a})CO₂R⁴, and —N(R^{a})SO₂R⁴;
R² is aryl optionally substituted by 1-3 substituents selected from R⁷, or heteroaryl optionally substituted by 1-3 substituents selected from R⁷;
R³ has the formula -L-Ar^{a}-N(R^{a})SO₃—R^{b}, wherein L is a bond, —(CR^{a}R^{b})ₙ—, —C(O)—, —C(O)N(R^{a})—, —(CR^{a}R^{b})ₙ—C(O)N(R^{a})—, —C(O)N(R^{a})—(CR^{a}R^{b})ₙ—, —(CR^{a}R^{b})ₙ—O—; and wherein Ar³ is arylene optionally substituted by 1-3 substituents selected from R⁷, or heteroarylene optionally substituted by 1-3 substituents selected from R⁷;
each R⁴ is, independently, H, alkyl, or aryl, wherein alkyl and aryl are each independently substituted by 1-3 substituents selected from R⁷;
each R⁵ and each R⁶ are independently H, alkyl or aryl wherein alkyl and aryl are each independently substituted by 1-3 substituents selected from R⁷; or R⁵ and R⁶ together with the atom to which they are attached form a heterocyclic group which is optionally substituted by 1-3 substituents selected from R⁷;
each R⁷, independently, is H, oxo, CN, halogen, —CF₃, —CHF₂, —CHO, —OH, —NO₂, —SH, —OCF₃, alkyl, alkoxy, alkenyl, alkynyl, cycloalkyl, aryl, heteroaryl, heterocyclyl, —CO₂R^{a}, —O-alkyl, —O-alkenyl, —O-alkynyl, —O-cycloalkyl, —O-aryl, —O-heteroaryl, —O-heterocyclyl, —(CH₂)ₙ-alkyl, —(CH₂)ₙ-alkoxy, —(CH₂)ₙ-alkenyl, —(CH₂)ₙ-alkynyl, —(CH₂)ₙ-cycloalkyl, —(CH₂)ₙ-aryl, —(CH₂)ₙ-heteroaryl, —(CH₂)ₙ-heterocyclyl, —N(R^{a})-alkyl, —N(R^{a})-alkoxy, —N(R^{a})-alkenyl, —N(R^{a})-alkynyl, —N(R^{a})-cycloalkyl, —N(R^{a})-aryl, —N(R^{a})-heteroaryl, —N(R^{a})-heterocyclyl, —SOₘ-alkoxy, —SOₘ-alkenyl, —SOₘ-alkynyl, —SOₘ-cycloalkyl, —SOₘ-aryl, —SOₘ-heteroaryl, —SOₘ-heterocyclyl, —N(R^{a})C(O)-alkyl, —N(R^{a})C(O)-alkoxy, —N(R^{a})C(O)-alkenyl, —N(R^{a})C(O)-alkynyl, —N(R^{a})C(O)-cycloalkyl, —N(R^{a})C(O)-aryl, —N(R^{a})C(O)-heteroaryl, —N(R^{a})C(O)-heterocyclyl, —C(O)N(R^{a})-alkyl, —C(O)N(R^{a})-alkoxy, —C(O)N(R^{a})-alkenyl, —C(O)N(R^{a})-alkynyl, —C(O)N(R^{a})-cycloalkyl, —C(O)N(R^{a})-aryl, —C(O)N(R^{a})-heteroaryl, or —C(O)N(R^{a})-heterocyclyl;
each R^{a}, independently, is H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl;
each R^{b}, independently, is H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl;
each m, independently, is 0, 1, or 2;
each n, independently, is 0, 1, 2, 3, or 4;
or a pharmaceutically acceptable salt thereof.

2. The compound of claim 1, wherein R¹ is —NR⁵R⁶.

3. The compound of claim 2, wherein R¹ is —NH₂.

4. The compound of claim 1, wherein R² is furyl, thienyl, imidazolyl, phenyl, pyridyl, thiazolyl, pyrazolyl, triazolyl, pyrrolyl, or oxazolyl, each of which is optionally substituted by 1-3 substituents selected from R⁷.

5. The compound of claim 4, wherein R² is furyl, thienyl, phenyl, methylfuryl, or methoxyphenyl.

6. The compound of claim 1, wherein L is —CH₂—; and Ar³ is arylene.

7. The compound of claim 6, wherein Ar³ is phenylene, methylphenylene, or methoxyphenylene.

8. The compound of claim 1, wherein R¹ is —NR⁵R⁶; R² is furyl, thienyl, imidazolyl, phenyl, pyridyl, thiazolyl, pyrazolyl, triazolyl, pyrrolyl, or oxazolyl, each of which is optionally substituted by 1-3 substituents selected from R⁷; L is —CH₂—; and Ar³ is arylene optionally substituted by 1-3 substituents selected from R⁷.

9. The compound of claim 8, wherein R¹ is —NH₂; R² is furyl, thienyl, phenyl, methylfuryl, or methoxyphenyl; L is —CH₂—; and Ar³ is phenylene, methylphenylene, or methoxyphenylene.

10. The compound of claim 1 selected from the group consisting of:
4-[(5-amino-7-(furan-2-yl)-3H-[1,2,3]triazolo[4,5-d]pyrimidin-3-yl)methyl]-2-methylphenylsulfamic acid;
4-[(5-amino-7-(furan-2-yl)-3H-[1,2,3]triazolo[4,5-d]pyrimidin-3-yl)methyl]phenylsulfamic acid;
4-[(5-amino-7-phenyl,2,3]triazolo[4,5-d]-pyrimidin-3-yl)methyl]phenylsulfamic acid;
4-[(5-amino-7-(thiophen-2-yl)-3H-[1,2,3]triazolo[4,5-d]-pyrimidin-3-yl)methyl]phenylsulfamic acid;
4-[(5-amino-7-(2-methoxyphenyl)-3H-[1,2,3]triazolo[4,5-d]pyrimidin-3-yl)methyl]phenylsulfamic acid;
3-[(5-amino-7-(furan-2-yl)-3H-[1,2,3]triazolo[4,5-d]pyrimidin-3-yl)methyl]-phenylsulfamic acid;
3-[(5-amino-7-(thiophen-2-yl)-3H-[1,2,3]triazolo[4,5-d]pyrimidin-3-yl)methyl]-phenylsulfamic acid;
3-[(5-amino-7-(2-methoxyphenyl)-3H-[1,2,3]triazolo[4,5-d]-pyrimidin-3-yl)methyl]-phenylsulfamic acid;
3-[(5-amino-7-phenyl-3H-[1,2,3]triazolo[4,5-d]pyrimidin-3-yl)methyl]-phenylsulfamic acid;
4-[(5-amino-7-(2-methoxyphenyl)-3H-[1,2,3]triazolo[4,5-d]pyrimidin-3-yl)methyl]-2-methylphenylsulfamic acid;
4-[(5-amino-7-(thiophen-2-yl)-3H-[1,2,3]triazolo[4,5-d]-pyrimidin-3-yl)methyl]-2-methylphenylsulfamic acid;
4-[(5-amino-7-phenyl-3H-[1,2,3]triazolo[4,5-d]pyrimidin-3-yl)methyl]-2-methylphenylsulfamic acid;
5-[(5-amino-7-(thiophen-2-yl)-3H-[1,2,3]triazolo[4,5-d]pyrimidin-3-yl)methyl]-2-methylphenylsulfamic acid;
5-[(5-amino-7-(furan-2-yl)-3H-[1,2,3]triazolo[4,5-d]-pyrimidin-3-yl)methyl]-2-methylphenylsulfamic acid;
5-[(5-amino-7-phenyl-3H-[1,2,3]triazolo[4,5-d]-pyrimidin-3-yl)methyl]-2-methylphenylsulfamic acid;
5-[(5-amino-7-(2-methoxyphenyl)-3H-[1,2,3]triazolo[4,5-d]-pyrimidin-3-yl)methyl]-2-methylphenylsulfamic acid;
4-[(5-amino-7-(2-methoxyphenyl)-3H[1,2,3]triazolo[4,5-d]pyrimidin-3-yl)methyl]-2-methoxyphenylsulfamic acid;
4-[(5-amino-7-phenyl-3H-[1,2,3]triazolo[4,5-d]pyrimidin-3-yl)methyl]-2-methoxyphenylsulfamic acid;
4-[(5-amino-7-(thiophen-2-yl)-3H-[1,2,3]triazolo[4,5-d]pyrimidin-3-yl)methyl]-2-methoxyphenylsulfamic acid;
4-[(5-amino-7-(furan-2-yl)-3H-[1,2,3]triazolo[4,5-d]pyrimidin-3-yl)methyl]-2-methoxyphenylsulfamic acid;
4-[(5-amino-7-(thiophen-2-yl)-3H-[1,2,3]triazolo[4,5-d]-pyrimidin-3-yl)methyl]-3-methoxyphenylsulfamic acid;
4-[(5-amino-7-(furan-2-yl)-3H-[1,2,3]triazolo[4,5-d]pyrimidin-3-yl)methyl]-3-methoxyphenylsulfamic acid;
4-[(5-amino-7-phenyl-3H-[1,2,3]triazolo[4,5-d]pyrimidin-3-yl)methyl]-3-methoxyphenylsulfamic acid;
4-[(5-amino-7-(2-methoxyphenyl)-3H-[1,2,3]triazolo[4,5-d]pyrimidin-3-yl)methyl]-3-methoxyphenylsulfamic acid;
4-[(5-amino-7-(5-methylfuran-2-yl)-3H-[1,2,3]triazolo[4,5-d]-pyrimidin-3-yl)methyl]phenylsulfamic acid;
3-[(5-amino-7-(5-methylfuran-2-yl)-3H-[1,2,3]triazolo[4,5-d]pyrimidin-3-yl)methyl]phenylsulfamic acid;
5-[(5-amino-7-(5-methylfuran-2-yl)-3H-[1,2,3]triazolo[4,5-d]-pyrimidin-3-yl)methyl]-2-methylphenylsulfamic acid;
4-[(5-amino-7-(5-methylfuran-2-yl)-3H-[1,2,3]triazolo[4,5-d]-pyrimidin-3-yl)methyl]-2-methylphenylsulfamic acid;
4-[(5-amino-7-(5-methylfuran-2-yl)-3H-[1,2,3]triazolo[4,5-d]-pyrimidin-3-yl)methyl]-2-methoxyphenylsulfamic acid;
4-[(5-amino-7-(5-methylfuran-2-yl)-3H-[1,2,3]triazolo[4,5-d]pyrimidin-3-yl)methyl]-3-methoxyphenylsulfamic acid; and
pharmaceutically acceptable salts thereof.

11. A pharmaceutical composition comprising a pharmaceutically acceptable carrier and a compound of formula (I): 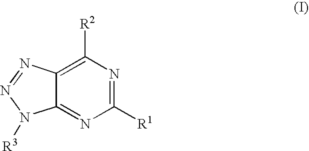
wherein
R¹ is selected from H, alkyl, aryl, heteroaryl, cycloalkyl, heterocyclyl, alkoxy, aryloxy, heteroaryloxy, alkylthio, arylthio, heteroarylthio, halogen, —CN, —NR⁵R⁶, —N(R^{a})C(O)R⁴, —N(R^{a})C(O)NR⁵R⁶, —N(R^{a})CO₂R⁴, and —N(R^{a})SO₂R⁴;
R² is aryl optionally substituted by 1-3 substituents selected from R⁷, or heteroaryl optionally substituted by 1-3 substituents selected from R⁷;
R³ has the formula -L-Ar^{a}-N(R^{a})SO₃—R^{b}, wherein L is a bond, —(CR^{a}R^{b})ₙ—, —C(O)—, —C(O)N(R^{a})—, -(CR^{a}R^{b})ₙ—C(O)N(R^{a})—, —C(O)N(R^{a})—(CR^{a}R^{b})ₙ—, —(CR^{a}R^{b})ₙ—O—; and wherein Ar³ is arylene optionally substituted by 1-3 substituents selected from R⁷, or heteroarylene optionally substituted by 1-3 substituents selected from R⁷;
each R⁴ is, independently, H, alkyl, or aryl, wherein alkyl and aryl are each independently substituted by 1-3 substituents selected from R⁷;
each R⁵ and each R⁶ are independently H, alkyl or aryl wherein alkyl and aryl are each independently substituted by 1-3 substituents selected from R⁷; or R⁵ and R⁶ together with the atom to which they are attached form a heterocyclic group which is optionally substituted by 1-3 substituents selected from R⁷;
each R⁷, independently, is H, oxo, CN, halogen, —CF₃, —CHF₂, —CHO, —OH, —NO₂, —SH, —OCF₃, alkyl, alkoxy, alkenyl, alkynyl, cycloalkyl, aryl, heteroaryl, heterocyclyl, —CO₂R^{a}, —O-alkyl, —O-alkenyl, —O-alkynyl, —O-cycloalkyl, —O-aryl, —O-heteroaryl, —O-heterocyclyl, —(CH₂)ₙ-alkyl, —(CH₂)ₙ-alkoxy, —(CH₂)ₙ-alkenyl, —(CH₂)ₙ-alkynyl, —(CH₂)ₙ-cycloalkyl, —(CH₂)ₙ-aryl, —(CH₂)ₙ-heteroaryl, —(CH₂)ₙ-heterocyclyl, —N(R^{a})-alkyl, —N(R^{a})-alkoxy, —N(R^{a})-alkenyl, —N(R^{a})-alkynyl, —N(R^{a})-cycloalkyl, —N(R^{a})-aryl, —N(R^{a})-heteroaryl, —N(R^{a})-heterocyclyl, —SOₘ-alkoxy, —SOₘ-alkenyl, —SOₘ-cycloalkyl, —SOₘ-aryl, —SOₘ-heteroaryl, —SOₘ-heterocyclyl, —N(R^{a})C(O)-alkyl, —N(R^{a})C(O)-alkoxy, —N(R^{a})C(O)-alkenyl, —N(R^{a})C(O)-alkynyl, —N(R^{a})C(O)-cycloalkyl, —N(R^{a})C(O)-aryl, —N(R^{a})C(O)-heteroaryl, —N(R^{a})C(O)-heterocyclyl, —C(O)N(R^{a})-alkyl, —C(O)N(R^{a})-alkoxy, —C(O)N(R^{a})-alkenyl, —C(O)N(R^{a})-alkynyl, —C(O)N(R^{a})-cycloalkyl, —C(O)N(R^{a})-aryl, —C(O)N(R^{a})-heteroaryl, or —C(O)N(R^{a})-heterocyclyl;
each R^{a}, independently, is H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl;
each R^{b}, independently, is H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl;
each m, independently, is 0, 1, or 2;
each n, independently, is 0, 1, 2, 3, or 4;
or a pharmaceutically acceptable salt thereof.

12. The composition of claim 11, wherein R¹ is —NR⁵R⁶.

13. The composition of claim 12, wherein R¹ is —NH₂.

14. The composition of claim 11, wherein R² is furyl, thienyl, imidazolyl, phenyl, pyridyl, thiazolyl, pyrazolyl, triazolyl, pyrrolyl, or oxazolyl, each of which is optionally substituted by 1-3 substituents selected from R⁷.

15. The composition of claim 14, wherein R² is furyl, thienyl, phenyl, methylfuryl, or methoxyphenyl.

16. The composition of claim 11, wherein L is —CH₂—; and Ar³ is arylene.

17. The composition of claim 16, wherein Ar³ is phenylene, methylphenylene, or methoxyphenylene.

18. The composition of claim 11, wherein R¹ is —NR⁵R⁶; R² is furyl, thienyl, imidazolyl, phenyl, pyridyl, thiazolyl, pyrazolyl, triazolyl, pyrrolyl, or oxazolyl, each of which is optionally substituted by 1-3 substituents selected from R⁷; L is —CH₂—; and Ar³ is arylene optionally substituted by 1-3 substituents selected from R⁷.

19. The composition of claim 18, wherein R¹ is —NH₂; R² is furyl, thienyl, phenyl, methylfuryl, or methoxyphenyl; L is —CH₂—; and Ar³ is phenylene, methylphenylene, or methoxyphenylene.

20. The composition of claim 11, wherein the compound is selected from the group consisting of:
4-[(5-amino-7-(furan-2-yl)-3H-[1,2,3]triazolo[4,5-d]pyrimidin-3-yl)methyl]-2-methylphenylsulfamic acid;
4-[(5-amino-7-(furan-2-yl)-3H-[1,2,3]triazolo[4,5-d]pyrimidin-3-yl)methyl]phenylsulfamic acid;
4-[(5-amino-7-phenyl,2,3]triazolo[4,5-d]pyrimidin-3-yl)methyl]phenylsulfamic acid;
4-[(5-amino-7-(thiophen-2-yl)-3H-[1,2,3]triazolo[4,5-d]-pyrimidin-3-yl)methyl]phenylsulfamic acid;
4-[(5-amino-7-(2-methoxyphenyl)-3H-[1,2,3]triazolo[4,5-d]pyrimidin-3-yl)methyl]phenylsulfamic acid;
3-[(5-amino-7-(furan-2-yl)-3H-[1,2,3]triazolo[4,5-d]-pyrimidin-3-yl)methyl]-phenylsulfamic acid;
3-[(5-amino-7-(thiophen-2-yl)-3H-[1,2,3]triazolo[4,5-d]pyrimidin-3-yl)methyl]-phenylsulfamic acid;
3-[(5-amino-7-(2-methoxyphenyl)-3H-[1,2,3]triazolo[4,5-d]pyrimidin-3-yl)methyl]-phenylsulfamic acid;
3-[(5-amino-7-phenyl-3H[1,2,3]triazolo[4,5-d]pyrimidin-3-yl)methyl]-phenylsulfamic acid;
4-[(5-amino-7-(2-methoxyphenyl)-3H-[1,2,3]triazolo[4,5-d]pyrimidin-3-yl)methyl]-2-methylphenylsulfamic acid;
4-[(5-amino-7-(thiophen-2-yl)-3H-[1,2,3]triazolo[4,5-d]-pyrimidin-3-yl)methyl]-2-methylphenylsulfamic acid;
4-[(5-amino-7-phenyl-3H-[1,2,3]triazolo[4,5-d]pyrimidin-3-yl)methyl]-2-methylphenylsulfamic acid;
5-[(5-amino-7-(thiophen-2-yl)-3H-[1,2,3]triazolo[4,5-d]-pyrimidin-3-yl)methyl]-2-methylphenylsulfamic acid;
5-[(5-amino-7-(furan-2-yl)-3H-[1,2,3]triazolo[4,5-d]-pyrimidin-3-yl)methyl]-2-methylphenylsulfamic acid;
5-[(5-amino-7-phenyl-3H-[1,2,3]triazolo[4,5-d]pyrimidin-3-yl)methyl]-2-methylphenylsulfamic acid;
5-[(5-amino-7-(2-methoxyphenyl)-3H-[1,2,3]triazolo[4,5-d]-pyrimidin-3-yl)methyl]-2-methylphenylsulfamic acid;
4-[(5-amino-7-(2-methoxyphenyl)-3H-[1,2,3]triazolo[4,5-d]-pyrimidin-3-yl)methyl]-2-methoxyphenylsulfamic acid;
4-[(5-amino-7-phenyl-3H-[1,2,3]triazolo[4,5-d]pyrimidin-3-yl)methyl]-2-methoxyphenylsulfamic acid;
4-[(5-amino-7-(thiophen-2-yl)-3H-[1,2,3]triazolo[4,5-d]-pyrimidin-3-yl)methyl]-2-methoxyphenylsulfamic acid;
4-[(5-amino-7-(furan-2-yl)-3H-[1,2,3]triazolo[4,5-d]-pyrimidin-3-yl)methyl]-2-methoxyphenylsulfamic acid;
4-[(5-amino-7-(thiophen-2-yl)-3H-[1,2,3]triazolo[4,5-d]pyrimidin-3-yl)methyl]-3-methoxyphenylsulfamic acid;
4-[(5-amino-7-(furan-2-yl)-3H-[1,2,3]triazolo[4,5-d]pyrimidin-3-yl)methyl]-3-methoxyphenylsulfamic acid;
4-[(5-amino-7-phenyl-3H-[1,2,3]triazolo[4,5-d]-pyrimidin-3-yl)methyl]-3-methoxyphenylsulfamic acid;
4-[(5-amino-7-(2-methoxyphenyl)-3H-[1,2,3]triazolo[4,5-d]pyrimidin-3-yl)methyl]-3-methoxyphenylsulfamic acid;
4-[(5-amino-7-(5-methylfuran-2-yl)-3H-[1,2,3]triazolo[4,5-d]pyrimidin-3-yl)methyl]phenylsulfamic acid;
3-[(5-amino-7-(5-methylfuran-2-yl)-3H-[1,2,3]triazolo[4,5-d]-pyrimidin-3-yl)methyl]phenylsulfamic acid;
5-[(5-amino-7-(5-methylfuran-2-yl)-3H-[1,2,3]triazolo[4,5-d]pyrimidin-3-yl)methyl]-2-methylphenylsulfamic acid;
4-[(5-amino-7-(5-methyl furan-2-yl)-3H-[1,2,3]triazolo[4,5-d]pyrimidin-3-yl)methyl]-2-methylphenylsulfamic acid;
4-[(5-amino-7-(5-methylfuran-2-yl)-3H-[1,2,3]triazolo[4,5-d]pyrimidin-3-yl)methyl]-2-methoxyphenylsulfamic acid;
4-[(5-amino-7-(5-methylfuran-2-yl)-3H-[1,2,3]triazolo[4,5-d]pyrimidin-3-yl)methyl]-3-methoxyphenylsulfamic acid; and
pharmaceutically acceptable salts thereof.

21. A method of treating a disorder comprising administering an effective dose of a compound, or a pharmaceutically acceptable salt thereof, to a subject in need of treatment of a disorder treatable by purine receptor blocking, wherein the compound has formula (I): 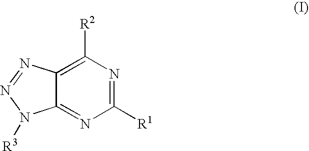
wherein
R¹ is selected from H, alkyl, aryl, heteroaryl, cycloalkyl, heterocyclyl, alkoxy, aryloxy, heteroaryloxy, alkylthio, arylthio, heteroarylthio, halogen, —CN, —NR⁵R⁶, —N(R^{a})C(O)R⁴, —N(R^{a})C(O)NR⁵R⁶, —N(R^{a})CO₂R⁴, and —N(R^{a})SO₂R⁴;
R² is aryl optionally substituted by 1-3 substituents selected from R⁷, or heteroaryl optionally substituted by 1-3 substituents selected from R⁷;
R³ has the formula -L-Ar³-N(R^{a})SO₃—R^{b}, wherein L is a bond, —(CR^{a}R^{b}), —C(O)—, —C(O)N(R^{a})—, —(CR^{a}R^{b})ⁿ—C(O)N(R^{a})—, —C(O)N(R^{a})—(CR^{a}R^{b})ₙ—, —(CR^{a}R^{b})—O—; and wherein Ar^{a} is arylene optionally substituted by 1-3 substituents selected from R⁷, or heteroarylene optionally substituted by 1-3 substituents selected from R⁷;
each R⁴ is, independently, H, alkyl, or aryl, wherein alkyl and aryl are each independently substituted by 1-3 substituents selected from R⁷;
each R⁵ and each R⁶ are independently H, alkyl or aryl wherein alkyl and aryl are each independently substituted by 1-3 substituents selected from R⁷; or R⁵ and R⁶ together with the atom to which they are attached form a heterocyclic group which is optionally substituted by 1-3 substituents selected from R⁷;
each R⁷, independently, is H, oxo, CN, halogen, CF₃, —CHO, —OH, —NO, —SH, —OCF₃, alkyl, alkoxy, alkenyl, alkynyl, cycloalkyl, aryl, heteroaryl, heterocyclyl, —CO₂R^{a}, —O-alkyl, —O-alkenyl, —O-alkynyl, —O-cycloalkyl, —O-aryl, —O-heteroaryl, —O-heterocyclyl, —(CH₂)ₙ-alkyl, —(CH₂)ₙ-alkoxy, —(CH₂)ₙ-alkenyl, —(CH₂)ₙ-alkynyl, —(CH₂)ₙ-cycloalkyl, —(CH₂)ₙ-aryl, —(CH₂)ₙ-heteroaryl, —(CH₂)ₙ-heterocyclyl, —N(R^{a})-alkyl, —N(R^{a})-alkoxy, —N(R^{a})-alkenyl, —N(R^{a})-alkynyl, —N(R^{a})-cycloalkyl, —N(R^{a})-aryl, —N(R^{a})-heteroaryl, —N(R^{a})-heterocyclyl, —SOₘ-alkyl, —SOₘ-alkoxy, —SOₘ-alkenyl, —SOₘ-alkynyl, —SOₘ-cycloalkyl, —SOₘ-aryl, —SOₘ-heteroaryl, —SOₘ-heterocyclyl, —N(R^{a})C(O)-alkyl, —N(R^{a})C(O)-alkoxy, —N(R^{a})C(O)-alkenyl, —N(R^{a})C(O)-alkynyl, —N(R^{a})C(O)-cycloalkyl, —N(R^{a})C(O)-aryl, —N(R^{a})C(O)-heteroaryl, —N(R^{a})C(O)-heterocyclyl, —C(O)N(R^{a})-alkyl, —C(O)N(R^{a})-alkoxy, —C(O)N(R^{a})-alkenyl, —C(O)N(R^{a})-alkynyl, —C(O)N(R^{a})-cycloalkyl, —C(O)N(R^{a})-aryl, —C(O)N(R^{a})-heteroaryl, or —C(O)N(R^{a})-heterocyclyl;
each R^{a}, independently, is H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl;
each R^{b}, independently, is H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl;
each m, independently, is 0, 1, or 2;
each n, independently, is 0, 1, 2, 3, or 4;
or a pharmaceutically acceptable salt thereof.

22. The method of claim 21, wherein the disorder is related to hyper functioning of purine receptors.

23. The method of claim 21, wherein the subject is in need of adenosine receptor blocking.

24. The method of claim 23, wherein the adenosine receptors are A_{2A} receptors.

25. The method of claim 24, wherein the disorder is a movement disorder.

26. The method of claim 25, wherein the movement disorder is Parkinson's disease.

27. The method of claim 26, wherein the movement disorder is drug-induced Parkinsonism, post-encephalitic Parkinsonism, Parkinsonism induced by poisoning or post-traumatic Parkinson's disease.

28. The method of claim 25, wherein the movement disorder is progressive supernuclear palsy, Huntingtons disease, multiple system atrophy, corticobasal degeneration, Wilsons disease, Hallerrorden-Spatz disease, progressive pallidal atrophy, Dopa-responsive dystonia-Parkinsonism, spasticity or other disorders of the basal ganglia which result in dyskinesias.

29. The method of claim 21, further comprising administering to the subject an additional drug useful in the treatment of movement disorders.

30. The method of claim 29, wherein the additional drug useful in the treatment of movement disorders is a drug useful in the treatment of Parkinson's disease.

31. The method of claim 30, wherein the additional drug is L-DOPA or a dopamine agonist.

32. The method of claim 21, wherein the disorder is depression, a cognitive or memory impairment disorder, acute or chronic pain, ADHD or narcolepsy.

33. The method of claim 32, wherein the cognitive or memory impairment disorder is Alzheimer's disease.

34. A method of making of compound comprising contacting a dithionite salt with a compound having the formula (I): 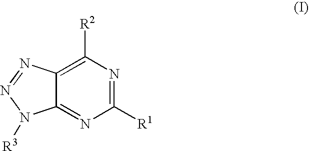
wherein
R¹ is selected from H, alkyl, aryl, heteroaryl, cycloalkyl, heterocyclyl, alkoxy, aryloxy, heteroaryloxy, alkylthio, arylthio, heteroarylthio, halogen, —CN, —NR⁵R⁶, —N(R^{a})C(O)R⁴, —N(R^{a})C(O)NR⁵R⁶, —N(R^{a})CO₂R⁴, and —N(R^{a})SO₂R⁴;
R² is aryl optionally substituted by 1-3 substituents selected from R⁷, or heteroaryl optionally substituted by 1-3 substituents selected from R⁷;
R³ has the formula -L-Ar^{a}-NO₂, wherein L is a bond, —(CR^{a}R^{b})ₙ—, —C(O)—, —C(O)N(R^{a})—, —(CR^{a}R^{b})ₙ—C(O)N(R^{a})—, —C(O)N(R^{a})—(CR^{a}R^{b})ₙ—, —(CR^{a}R^{b})ₙ—O—; and wherein Ar³ is arylene optionally substituted by 1-3 substituents selected from R⁷, or heteroarylene optionally substituted by 1-3 substituents selected from R⁷;
each R⁴ is, independently, H, alkyl, or aryl, wherein alkyl and aryl are each independently substituted by 1-3 substituents selected from R⁷;
each R⁵ and each R⁶ are independently H, alkyl or aryl wherein alkyl and aryl are each independently substituted by 1-3 substituents selected from R⁷; or R⁵ and R⁶ together with the atom to which they are attached form a heterocyclic group which is optionally substituted by 1-3 substituents selected from R⁷;
each R⁷, independently, is H, oxo, CN, halogen, —CF₃, —CHF₂, —CHO, —OH, —NO₂, —SH, —OCF₃, alkyl, alkoxy, alkenyl, alkynyl, cycloalkyl, aryl, heteroaryl, heterocyclyl, —CO₂R^{a}, —O-alkyl, —O-alkenyl, —O-alkynyl, —O-cycloalkyl, —O-aryl, —O-heteroaryl, —O-heterocyclyl, —(CH₂)ₙ-alkyl, —(CH₂)ₙ-alkoxy, —(CH₂)ₙ-alkenyl, —(CH₂)ₙ-alkynyl, —(CH₂)ₙ-cycloalkyl, —(CH₂)ₙ-aryl, —(CH₂)ₙ-heteroaryl, —(CH₂)ₙ-heterocyclyl, —N(R^{a})-alkyl, —N(R^{a})-alkoxy, —N(R^{a})-alkenyl, —N(R^{a})-alkynyl, —N(R^{a})-cycloalkyl, —N(R^{a})-aryl, —N(R^{a})-heteroaryl, —N(R^{a})-heterocyclyl, —SOₘ-alkoxy, —SOₘ-alkenyl, —SOₘ-alkynyl, —SOₘ-cycloalkyl, —SOₘ-aryl, —SOₘ-heteroaryl, —SOₘ-heterocyclyl, —N(R^{a})C(O)-alkyl, —N(R^{a})C(O)-alkoxy, —N(R^{a})C(O)-alkenyl, —N(R^{a})C(O)-alkynyl, —N(R^{a})C(O)-cycloalkyl, —N(R^{a})C(O)-aryl, —N(R^{a})C(O)-heteroaryl, —N(R^{a})C(O)-heterocyclyl, —C(O)N(R^{a})-alkyl, —C(O)N(R^{a})-alkoxy, —C(O)N(R^{a})-alkenyl, —C(O)N(R^{a})-alkynyl, —C(O)N(R^{a})-cycloalkyl, —C(O)N(R^{a})-aryl, —C(O)N(R^{a})-heteroaryl, or —C(O)N(R^{a})-heterocyclyl;
each R^{a}, independently, is H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl;
each R^{b}, independently, is H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl;
each m, independently, is 0, 1, or 2;
each n, independently, is 0, 1, 2, 3, or 4;
or a pharmaceutically acceptable salt thereof.

35. A method of making of compound comprising contacting chlorosulfonic acid with a compound having the formula (I): 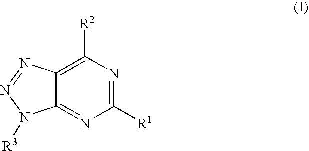
wherein
R¹ is selected from H, alkyl, aryl, heteroaryl, cycloalkyl, heterocyclyl, alkoxy, aryloxy, heteroaryloxy, alkylthio, arylthio, heteroarylthio, halogen, —CN, —NR⁵R⁶, —N(R^{a})C(O)R⁴, —N(R^{a})C(O)NR⁵R⁶, —N(R^{a})CO₂R⁴, and —N(R^{a})SO₂R⁴;
R² is aryl optionally substituted by 1-3 substituents selected from R⁷, or heteroaryl optionally substituted by 1-3 substituents selected from R⁷;
R³ has the formula -L-Ar³—NH₂, wherein L is a bond, —(CR^{a}R^{b})ₙ—, —C(O)—, —C(O)N(R^{a})—, —(CR^{a}R^{b})ₙ—C(O)N(R^{a})—, —C(O)N(R^{a})—(CR^{a}R^{b})ₙ—, —(CR^{a}R^{b})ₙ—O—; and wherein Ar³ is arylene optionally substituted by 1-3 substituents selected from R⁷, or heteroarylene optionally substituted by 1-3 substituents selected from R⁷;
each R⁴ is, independently, H, alkyl, or aryl, wherein alkyl and aryl are each independently substituted by 1-3 substituents selected from R⁷;
each R⁵ and each R⁶ are independently H, alkyl or aryl wherein alkyl and aryl are each independently substituted by 1-3 substituents selected from R⁷; or R⁵ and R⁶ together with the atom to which they are attached form a heterocyclic group which is optionally substituted by 1-3 substituents selected from R⁷;
each R⁷, independently, is H, oxo, CN, halogen, —CF₃, —CHF₂, —CHO, —OH, —NO₂, —SH, —OCF₃, alkyl, alkoxy, alkenyl, alkynyl, cycloalkyl, aryl, heteroaryl, heterocyclyl, —CO₂R^{a}, —O-alkyl, —O-alkenyl, —O-alkynyl, —O-cycloalkyl, —O-aryl, —O-heteroaryl, —O-heterocyclyl, —(CH₂)ₙ-alkyl, —(CH₂)ₙ-alkoxy, —(CH₂)ₙ-alkenyl, —(CH₂)ₙ-alkynyl, —(CH₂)ₙ-cycloalkyl, —(CH₂)ₙ-aryl, —(CH₂)ₙ-heteroaryl, —(CH₂)ₙ-heterocyclyl, —N(R^{a})-alkyl, —N(R^{a})-alkoxy, —N(R^{a})-alkenyl, —N(R^{a})-alkynyl, —N(R^{a})-cycloalkyl, —N(R^{a})-aryl, —N(R^{a})-heteroaryl, —N(R^{a})-heterocyclyl, —SOₘ-alkoxy, —SOₘ-alkenyl, —SOₘ-alkynyl, —SOₘ-cycloalkyl, —SOₘ-aryl, —SOₘ-heteroaryl, —SOₘ-heterocyclyl, —N(R^{a})C(O)-alkyl, —N(R^{a})C(O)-alkoxy, —N(R^{a})C(O)-alkenyl, —N(R^{a})C(O)-alkynyl, —N(R^{a})C(O)-cycloalkyl, —N(R^{a})C(O)-aryl, —N(R^{a})C(O)-heteroaryl, —N(R^{a})C(O)-heterocyclyl, —C(O)N(R^{a})-alkyl, —C(O)N(R^{a})-alkoxy, —C(O)N(R^{a})-alkenyl, —C(O)N(R^{a})-alkynyl, —C(O)N(R^{a})-cycloalkyl, —C(O)N(R^{a})-aryl, —C(O)N(R^{a})-heteroaryl, or —C(O)N(R^{a})-heterocyclyl;
each R^{a}, independently, is H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl;
each R^{b}, independently, is H, halogen, C₁-C₆ alkyl, C₃-C₈ cycloalkyl, phenyl or benzyl, each of which is optionally substituted with —OH, halo, —CF₃, —CN, —NO₂, oxo, alkyl, alkoxy or cycloalkyl;
each m, independently, is 0, 1, or 2;
each n, independently, is 0, 1, 2, 3, or 4;
or a pharmaceutically acceptable salt thereof.

36. The method of claim 34, wherein R¹ is —NR⁵R⁶.

37. The method of claim 36, wherein R¹ is —NH₂.

38. The method of claim 31, wherein R² is furyl, thienyl, imidazolyl, phenyl, pyridyl, thiazolyl, pyrazolyl, triazolyl, pyrrolyl, or oxazolyl, each of which is optionally substituted by 1-3 substituents selected from R⁷.

39. The method of claim 38, wherein R² is furyl, thienyl, phenyl, methylfuryl, or methoxyphenyl.

40. The method of claim 31, wherein L is —CH₂—; and Ar³ is arylene.

41. The method of claim 40, wherein Ar³ is phenylene, methylphenylene, or methoxyphenylene.

42. The method of claim 34, wherein Rⁱ is —NR⁵R⁶; R² is furyl, thienyl, imidazolyl, phenyl, pyridyl, thiazolyl, pyrazolyl, triazolyl, pyrrolyl, or oxazolyl, each of which is optionally substituted by 1-3 substituents selected from R⁷; L is —CH₂—; and Ar³ is arylene optionally substituted by 1-3 substituents selected from R⁷.

43. The method of claim 42, wherein R¹ is —NH₂; R² is furyl, thienyl, phenyl, methylfuryl, or methoxyphenyl; L is —CH₂—; and Ar³ is phenylene, methylphenylene, or methoxyphenylene.

44. The method of claim 34, wherein the compound of formula (I) is selected from the group consisting of:
7-(furan-2-yl)-3-(3-methyl-4-nitrobenzyl)-3H-[1,2,3]triazolo[4,5-d]pyrimidin-5-amine;
7-(furan-2-yl)-3-(4-nitrobenzyl)-3H-[1,2,3]triazolo[4,5-d]pyrimidin-5-amine;
3-(4-nitrobenzyl)-7-(phenyl)-3H-[1,2,3]triazolo[4,5-d]pyrimidin-5-amine;
3-(4-nitrobenzyl)-7-(thiophen-2-yl)-3H-[1,2,3]triazolo[4,5-d]pyrimidin-5-amine;
7-(2-methoxyphenyl)-3-(4-nitrobenzyl)-3H-[1,2,3]triazolo[4,5-d]pyrimidin-5-amine;
7-(furan-2-yl)-3-(3-nitrobenzyl)-3H-[1,2,3]triazolo[4,5-d]pyrimidin-5-amine;
3-(3-nitrobenzyl)-7-(thiophen-2-yl)-3H-[1,2,3]triazolo[4,5-d]pyrimidin-5-amine;
7-(2-methoxyphenyl)-3-(3-nitrobenzyl)-3H-[1,2,3]triazolo[4,5-d]pyrimidin-5-amine;
3-(3-nitrobenzyl)-7-phenyl-3H-[1,2,3]triazolo[4,5-d]pyrimidin-5-amine;
7-(2-methoxyphenyl)-3-(3-methyl-4-nitrobenzyl)-3H-[1,2,3]triazolo[4,5-d]pyrimidin-5-amine;
3-(3-methyl-4-nitrobenzyl)-7-(thiophen-2-yl)-3H-[1,2,3]triazolo[4,5-d]pyrimidin-5-amine;
3-(4-methyl-3-nitrobenzyl)-7-(thiophen-2-yl)-3H-[1,2,3]triazolo[4,5-d]pyrimidin-5-amine;
7-(furan-2-yl)-3-(4-methyl-3-nitrobenzyl)-3H-[1,2,3]triazolo[4,5-d]pyrimidin-5-amine;
3-(4-methyl-3-nitrobenzyl)-7-phenyl-3H-[1,2,3]triazolo[4,5-d]pyrimidin-5-amine;
7-(2-methoxyphenyl)-3-(4-methyl-3-nitrobenzyl)-3H-[1,2,3]triazolo[4,5-d]pyrimidin-5-amine;
3-(3-methoxy-4-nitrobenzyl)-7-(2-methoxyphenyl)-3H-[1,2,3]triazolo[4,5-d]pyrimidin-5-amine;
3-(3-methoxy-4-nitrobenzyl)-7-phenyl-3H-[1,2,3]triazolo[4,5-d]pyrimidin-5-amine;
3-(3-methoxy-4-nitrobenzyl)-7-(thiophen-2-yl)-3H-[1,2,3]triazolo[4,5-d]pyrimidin-5-amine;
7-(furan-2-yl)-3-(3-methoxy-4-nitrobenzyl)-3H-[1,2,3]triazolo[4,5-d]pyrimidin-5-amine;
3-(2-methoxy-4-nitrobenzyl)-7-(thiophen-2-yl)-3H-[1,2,3]triazolo[4,5-d]pyrimidin-5-amine;
7-(furan-2-yl)-3-(2-methoxy-4-nitrobenzyl)-3H-[1,2,3]triazolo[4,5-d]pyrimidin-5-amine;
3-(2-methoxy-4-nitrobenzyl)-7-phenyl-3H-[1,2,3]triazolo[4,5-d]pyrimidin-5-amine;
3-(2-methoxy-4-nitrobenzyl)-7-(2-methoxyphenyl)-3H-[1,2,3]triazolo[4,5-d]pyrimidin-5-amine;
7-(5-methylfuran-2-yl)-3-(4-nitrobenzyl)-3H-[1,2,3]triazolo[4,5-d]pyrimidin-5-amine;
7-(5-methylfuran-2-yl)-3-(3-nitrobenzyl)-3H-[1,2,3]triazolo[4,5-d]pyrimidin-5-amine;
3-(4-methyl-3-nitrobenzyl)-7-(5-methylfuran-2-yl)-3H-[1,2,3]triazolo[4,5-d]pyrimidin-5-amine;
3-(3-methyl-4-nitrobenzyl)-7-(5-methylfuran-2-yl)-3H-[1,2,3]triazolo[4,5-d]pyrimidin-5-amine;
3-(3-methoxy-4-nitrobenzyl)-7-(5-methylfuran-2-yl)-3H-[1,2,3]triazolo[4,5-d]pyrimidin-5-amine; and
3-(2-methoxy-4-nitrobenzyl)-7-(5-methylfuran-2-yl)-3H-[1,2,3]triazolo[4,5-d]pyrimidin-5-amine.
